(19) United States
(12) Patent Application Publication
(10) Pub. No.: US 2020/0087665 A1
(43) Pub. Date: Mar. 19, 2020

(54) SYSTEMS

(71) Applicant: New York Stem Cell Foundation, Inc., New York, NY (US)

(72) Inventors: Malin Parmar, Lund (SE); Johan Jakobsson, Malmoe (SE); Janelle Drouin-Ouellet, Lund (SE); Shong Lau, La Jolla, CA (US)

(21) Appl. No.: 16/610,787

(22) PCT Filed: May 11, 2018
(86) PCT No.: PCT/EP2018/062261

§ 371 Date: Nov. 4, 2019

(30) Foreign Application Priority Data
May 12, 2017 (SE) 1730131-8

(51) Int. Cl. C12N 15/113; C12N 15/86; C12N 5/0793; G01N 33/50; C12Q 1/6883

(52) U.S. Cl. CPC C12N 15/113; C12Q 2600/112; C12N 5/0619; G01N 33/5058; C12Q 1/6883; C12N 2310/531; C12N 2310/14; C12N 2740/15043; C12N 2506/1307; C12N 2501/10; C12N 2501/999; C12N 2510/00; C12Q 2600/178; C12Q 2600/118; C12Q 2600/158; C12N 15/86

(57) ABSTRACT

An optimized method based on a dual promoter vector of the reprogramming factors combined with knock-down of the neural silencing complex RESTi to convert adult fibroblasts into induced neurons (iNs). We have also designed and cloned vector constructs of which some include all these components which allows for a one-step method to efficiently reprogram dermal fibroblasts including those obtained from elderly individuals. The single vector system can be used to obtain iNs of high yield and purity from biopsies from aged individuals with a range of familial and sporadic neurodegenerative disorders including Parkinson's, Huntington's as well as Alzheimer's disease.

The present invention relates to gene expression systems and in particular to gene expression systems for use in obtaining induced neurons from adult fibroblast cells.

New advances in somatic cell reprogramming offers unique access to human neurons from defined patient groups for modeling neurological disorders in vitro. This has enabled a number of mechanistic studies to better understand how pathology arises and develops, and also creates new opportunities for early and differential diagnostic tests and drug screens.

As an alternative for generating disease and patient specific neurons, adult fibroblasts can be directly converted into functional neurons using chemicals, defined sets of transcription factors or microRNAs (miRNAs) for chemical reprogramming. This type of direct reprograming allows fibroblasts to be converted into induced neurons (iNs) without transitioning via a proliferative stem cell intermediate, making the process faster and easier. In addition, recent studies have also demonstrated that the resulting iNs, unlike induced pluripotent stem cells (iPSCs), maintain the ageing signature of the donor, making iNs ideal candidates for modeling neuronal pathology in late-onset diseases.

However, a number of factors such as species and age of donor, passage number and prolonged culturing of cells prior to conversion limits the reprogramming efficiency of this approach. In particular, human cells are harder to reprogram than rodent cells, cells from adult donors are much harder to reprogram than fetal cells, and in vitro expansion and/or extensive culturing and passaging of cells prior to conversion often prevents successful conversion. The reason for these differences is not fully understood, but the fact that human fibroblasts from aged individuals are more resistant/refractory to reprogramming than fetal fibroblasts creates a barrier for using these cells for large scale biomedical applications and future clinical applications.

We have identified the RE1-silencing transcription factor (REST) complex as a potential barrier to reprogramming of adult human fibroblasts. We confirm this by showing that REST inhibition (RESTi), when combined with the neural conversion genes Ascl1 and Brn2 can remove the reprogramming barrier in adult dermal and lung fibroblasts and yield a high number of functionally mature neurons. This high-level conversion is maintained over extensive passaging of the fibroblasts.

Further, we constructed an all-in-one neural conversion vector that contains all the components necessary for robust, high yield neural conversion of adult dermal fibroblasts. We then demonstrated that such a vector could be used to efficiently convert fibroblasts collected at three different clinical sites from individuals with idiopathic as well as genetic forms of Parkinson's disease and Alzheimer's disease as well as patients with Huntington's disease. This new approach to iN conversion has great potential for disease modeling, diagnostics and drug screening and discovery across a range of neurological disorders that develop later in life—a set of conditions that have to date been nearly impossible to model using this approach.

Accordingly, a first aspect of the invention provides a gene expression system comprising

a. at least one nucleotide sequence encoding a neuronal conversion factor; and

b. at least one nucleotide sequence encoding a REST-silencing sequence capable of suppressing REST-expression.

By a “gene expression system” we include the meaning of one or more genes to be expressed together with any other one or more nucleic acid molecules which are required for expression of the one or more genes. Expression systems typically include one or more regulatory sequences upstream and/or downstream of the coding sequence. Preferably, the one or more regulatory sequences are operably linked to the one or more genes to be expressed.

For example, transcription factors recognise and bind to transcriptional regulatory sequences and control the production of a message transcribed from the gene. Transcriptional regulatory nucleic acid sequences involved in the regulation of gene expression include promoters, enhancers, and regulatory sequences to which transcription factors or transcriptional regulatory proteins bind, and which are required for initiation of transcription. Other regulatory sequences may include signals of initiation and termination of translation or other translational regulatory sequences. Thus, in addition to one or more coding sequences, the gene expression system may include one or more regulatory elements (e.g. a promoter, an enhancer, a regulatory sequence to which a transcription factor and/or transcription regulatory protein binds, a signal of initiation and a signal of termination fo translation). Such regulatory elements are well known in the art and can be selected to optimise expression of the one or more genes in a given host cell

By “gene” we include the meaning of any nucleic acid sequence which is capable of being transcribed into a protein or peptide of interest. The gene may include both coding and non-coding regions, or it may include only coding regions. In other words, the gene may include only exons (eg the coding sequence), or it may include exons and introns.

By “neuronal conversion factor”, we include the meaning of any gene product or molecule that induces conversion of a non-neuronal cell into a cell with neuron-like properties, i.e. an induced neuron cell. Thus, the neuronal conversion factor may convert or reprogram a cell that is not characterised as a neuron (e.g. based on a combination of morphology and function) into a cell that has one or more neuron-like properties. This may also be considered as differentiation of the non-neuronal cell into an induced neuron cell. It is preferred if the neuronal conversion factor converts the non-neuronal cell into a neuron-like cell or induced neuron without transitioning via a proliferative stem cell intermediate.

For the avoidance of doubt, we include neuronal conversion factors that partially or completely differentiate a non-neuronal cell into a cell with neuron-like properties. Thus, the neuronal conversion factor may be one that induces a non-neuronal cell to have only one neuron-like property that was not previously present, or the neuronal conversion factor may be one that induces a non-neuronal cell to have more than one neuron-like property that was not previously present, such as at least 2, 3, 4 or 5 neuronal-like properties, or as many neuron-like properties which mean that the cell is determined to be a neuron-like cell based on a combination of morphological and functional tests.

By “neuron-like properties” we include the meaning of properties normally attributed to neurons/nerve cells such as morphological properties (for example neurite outgrowth, the presence of a soma/cell body, dendrites, axon and/or synapses); expression of neuronal specific markers such as MAP2, bIII-Tubulin, NeuN, Synapsin and Tau; excitatory or inhibitory membrane properties, for example as evidence by expression of vGlut and/or Gad67; and membrane depolarization capacity, for example as measured in a patch-clamp assay. Such properties can be determined using well established methods in the art and as described further in the Examples. For example, neuron-like morphology can be assessed using microscopy, neuron specific markers can be assessed using immunofluorescence or gene expression analysis, and functional properties can be assessed by patch clamp electrophysiology or functional imaging. Suitable techniques that can be used to characterise cells as being “neuron-like cells” also include those described in Drouin-Ouellet et al (2017) Front Neurosci 11:530, the entire contents of which are incorporated herein by reference.

Typically, the neuronal conversion factor, as well as inducing or upregulating one or more neuron-like properties, will downregulate one or more properties that are attributed to the non-neuronal cell. Again, such properties may be morphological properties, expression of specific markers of the non-neuronal cell, and/or functional properties of the non-neuronal cell. Such properties, and techniques to assess them, are well known in the art. For example, properties of fibroblasts include morphological properties and marker gene expression, e.g. collagen and/or immunoreactivity with anti-fibroblast antibody clone TE-7 (e.g. Merck catalogue number CBL271).

The neuronal conversion factor can be any molecule such as any of a peptide, a protein, a peptidomimetic, a nucleic acid, a microRNA, a natural product, a synthetic product, a carbohydrate, an aptamer or a small molecule. For example, the neuronal conversion factor may be a transcription factor, signalling molecule or a microRNA known to be involved in neuronal lineage determination during development of cell fate regulation. Preferably, the neuronal conversion factor is a nucleic acid or a small molecule. Examples of neuronal conversion factors include transcription factors, small molecules, microRNAs, small hairpin RNAs (shRNAs) and short interfering RNAs (siRNAs). Particular examples of neuronal conversion factors include: those listed in the “Reprogramming strategy” column of Table 1 of Drouin-Ouellet et al 2017 Front Neurosci; Y-27632; SP600125; Repsox; G06983; FoxA2; Lmx1a; Lmx1b; Otx2.

In the context of the present invention, it will be appreciated that the neuronal conversion factor must be capable of being encoded by at least one nucleotide sequence.

In a preferred embodiment, the neuronal conversion factor is selected from the group consisting of ASCL1 and BRN2. Thus, it will be appreciated that the invention provides a gene expression system comprising

a. (i) a nucleotide sequence encoding ASCL1;

(ii) a nucleotide sequence encoding BRN2; and

b. at least one nucleotide sequence encoding a REST-silencing sequence capable of suppressing REST-expression.

By ASCL1 or ASCL1 peptide we include the meaning of Achaete-scute homolog 1 (ASCL1, also known as Ashl, hASH-1, bHLHa46 or basic-helix-loop-helix protein 46) which is a 25 kDa basic helix-loop-helix (bHLH) protein. The amino acid sequence of human ASCL1 is

(NP_004307.2)
(SEQ ID NO: 1)
MESSAKMESG GAGQQPQPQP QQPFLPPAAC FFATAAAAAA
AAAAAAAQSA QQQQQQQQQQ QQAPQLRPAA DGQPSGGGHK
SAPKQVKRQR SSSPELMRCK RRLNFSGFGY SLPQQQPAAV
ARRNERERNR VKLVNLGFAT LREHVPNGAA NKKMSKVETL
RSAVEYIRAL QQLLDEHDAV SAAFQAGVLS PTISPNYSND
LNSMAGSPVS SYSSDEGSYD PLSPEEQELL DFTNWF

and so in one embodiment the ASCL1 is human ASCL1. We also include the meaning of orthologs of ASCL1 derived from other species, for example mammalian species such as mouse (NP_032579), chimpanzee (XP_009424458), cynomolgus monkey (XP_005572101), and rat (NP_032579).

By BRN2 or BRN2 peptide we include the meaning of Brain-2 (BRN2, also known as Brain-specific homeobox/POU domain protein 2, POU3F2, nervous system-specific octamer-binding transcription factor N-Oct-3, octamer-binding protein 7, Oct-7, or Octamer-binding transcription factor 7 (OTF-7)). The amino acid sequence of human BRN2 is

(NP_005595.2)
(SEQ ID NO: 2)
MATAASNHYS LLTSSASIVH AEPPGGMQQG AGGYREAQSL
VQGDYGALQS NGHPLSHAHQ WITALSHGGG GGGGGGGGGG
GGGGGGGGDG SPWSTSPLGQ PDIKPSVVVQ QGGRGDELHG
PGALQQQHQQ QQQQQQQQQQ QQQQQQQQQR PPHLVHHAAN
HHPGPGAWRS AAAAAHLPPS MGASNGGLLY SQPSFTVNGM
LGAGGQPAGL HHHGLRDAHD EPHHADHHPH PHSHPHQQPP
PPPPPQGPPG HPGAHHDPHS DEDTPTSDDL EQFAKQFKQR
RIKLGFTQAD VGLALGTLYG NVFSQTTICR FEALQLSFKN
MCKLKPLLNK WLEEADSSSG SPTSIDKIAA QGRKRKKRTS
IEVSVKGALE SHFLKCPKPS AQEITSLADS LQLEKEVVRV
WFCNRRQKEK RMTPPGGTLP GAEDVYGGSR DTPPHHGVQT
PVQ

and so in one embodiment the BRN2 is human BRN2. We also include the meaning of orthologs of BRN2 derived from other species for example mammalian species such as mouse (NP_032925).

It is well known that certain polypeptides are polymorphic, and so it is appreciated that some natural variation of the sequences of ASCL1 and BRN2 outlined above may occur. Thus, also included are naturally occurring variants of human ASCL1 or orthologs thereof, and naturally occurring variants of human BRN2 or orthologs thereof, in which one or more of the amino acid residues have been replaced with another amino acid.

We also include functional variants of ASCL1 and BRN2. By functional variant we include the meaning of a variant of the protein (e.g. ASCL1 or BRN2) which retains at least one activity of the protein (eg ASCL1 or BRN2) e.g. the ability to act as a neuronal conversion factor. Variations include insertions, deletions and substitutions, either conservative or non-conservative. By “conservative substitutions” is intended combinations such as Gly, Ala; Val, Ile, Leu; Asp, Glu; Asn, Gln; Ser, Thr; Lys, Arg; and Phe, Tyr. The functional variants include variants of human ASCL1 or any orthologue thereof. Tests for assessing whether or not the variant retains the activity of a neuronal conversion factor are known in the art, and include those described above. Preferably a functional variant of ASCL1 and/or BRN2 is capable of converting fibroblasts into neurons.

It is preferred if the functional variant of ASCL1 has at least 60% sequence identity to the amino acid sequence of human ASCL1 (SEQ ID NO: 1), such as at least 70%, 75%, 80%, 85%, 90%, 95%, or 99% sequence identity.

Similarly, it is preferred if the functional variant of BRN2 has at least 60% sequence identity to the amino acid sequence of human BRN2 (SEQ ID NO: 2), such as at least 70%, 75%, 80%, 85%, 90%, 95%, or 99% sequence identity.

By ASCL1, ASCL1 peptide, BRN2 or BRN2 peptide, we also include the meaning of portions of the full-length ASCL1 or BRN2 proteins, or variants thereof, that nevertheless retain the ability to act as neuronal conversion factors. Tests for assessing whether or not the variant retains the activity of a neuronal conversion factor are known in the art, and include those described above. The portion of ASCL1 may comprise at least 20, 30, 40, 50, 100, 150 or 200 consecutive amino acids of the full-length ASCL1 proteins or variants mentioned above, such as human ASCL1 (SEQ ID NO: 1). The portion of BRN2 may comprise at least 20, 30, 40, 50, 100, 150, 200, 250, 300, 350, or 400 consecutive amino acids of the full-length ASCL1 proteins or variants mentioned above, such as human ASCL1 (SEQ ID NO: 1). Also included are portions of ASCL1 and BRN2 (eg human ASCL1 and BRN2) in which one or more amino acid residues are substituted, such as up to 2, 3, 4, 5, 6, 7, 8, 9 or 10 amino acid residues. Thus, it will be appreciated that variants of portions of ASCL1 or BRN2 are also included.

By REST we include the meaning of RE1-silencing transcription factor (also known as neuron-restrictive silencer factor (NRSF) XBR, REST4, WT6, GINGF5 and HGF5), which acts as a transcriptional repressor. The amino acid sequence of human REST is:

(NP_001180437)
(SEQ ID NO: 3)
MATQVMGQSSGGGGLFTSSGNIGMALPNDMYDLHDLSKAELAAPQLIMLAN
VALTGEVNGSCCDYLVGEERQMAELMPVGDNNFSDSEEGEGLEESADIKGE
PHGLENMELRSLELSVVEPQPVFEASGAPDIYSSNKDLPPETPGAEDKGKS
SKTKPFRCKPCQYEAESEEQFVHHIRVHSAKKFFVEESAEKQAKARESGSS
TAEEGDFSKGPIRCDRCGYNTNRYDHYTAHLKHHTRAGDNERVYKCIICTY
TTVSEYHWRKHLRNHFPRKVYTCGKCNYFSDRKNNYVQHVRTHTGERPYKC
ELCPYSSSQKTHLTRHMRTHSGEKPFKCDQCSYVASNQHEVTRHARQVHNG
PKPLNCPHCDYKTADRSNFKKHVELHVNPRQFNCPVCDYAASKKCNLQYHF
KSKHPTCPNKTMDVSKVKLKKTKKREADLPDNITNEKTEIEQTKIKGDVAG
KKNEKSVKAEKRDVSKEKKPSNNVSVIQVTTRTRKSVTEVKEMDVHTGSNS
EKFSKTKKSKRKLEVDSHSLHGPVNDEESSTKKKKKVESKSKNNSQEVPKG
DSKVEENKKQNTCMKKSTKKKTLKNKSSKKSSKPPQKEPVEKGSAQMDPPQ
MGPAPTEAVQKGPVQVEPPPPMEHAQMEGAQIRPAPDEPVQMEVVQEGPAQ
KELLPPVEPAQMVGAQIVLAHMELPPPMETAQTEVAQMGPAPMEPAQMEVA
QVESAPMQVVQKEPVQMELSPPMEVVQKEPVQIELSPPMEVVQKEPVKIEL
SPPIEVVQKEPVQMELSPPMGVVQKEPAQREPPPPREPPLHMEPISKKPPL
RKDKKEKSNMQSERARKEQVLIEVGLVPVKDSWLLKESVSTEDLSPPSPPL
PKENLREEASGDQKLLNTGEGNKEAPLQKVGAEEADESLPGLAANINESTH
ISSSGQNLNTPEGETLNGKHQTDSIVCEMKMDTDQNTRENLTGINSTVEEP
VSPMLPPSAVEEREAVSKTALASPPATMAANESQEIDEDEGIHSHEGSDLS
DNMSEGSDDSGLHGARPVPQESSRKNAKEALAVKAAKGDFVCIFCDRSFRK
GKDYSKHLNRHLVNVYYLEEAAQGQE

and so in one embodiment the REST is human REST.

We also include the meaning of orthologs of REST derived from other species for example mammalian species such as mouse (NP_035393.2) and rat (NP_113976.1). Also included are natural and functional variants of REST that share REST activity, e.g. transcription repression activity.

By a REST-silencing sequence capable of suppressing REST-expression we include the meaning of any nucleotide sequence, typically RNA, that reduces the level of transcription and/or translation of REST. By reduces the level of transcription and/or translation of REST, we include the meaning of reducing the level of transcription and/or translation of REST to less than 90% of the level of transcription and/or translation of REST apparent in the absence of the REST-silencing sequence, such as less than 80%, 70%, or 60%, and preferably less than 50%, 40%, 30%, 20% or 10% of the level of transcription and/or translation of REST apparent in the absence of the REST-silencing sequence, and most preferably to an undetectable level of transcription and/or translation of REST. Any suitable method of determining the level of transcription and/or translation of REST can be used as is known in the art, such as PCR (e.g. qRT-PCR) as described further in the examples.

For the avoidance of doubt, it will be appreciated that by “at least one nucleotide sequence encoding a REST-silencing sequence capable of suppressing REST-expression” we include the meaning of the nucleotide sequence being the REST-silencing sequence itself and the nucleotide sequence being capable of being converted into the REST-silencing sequence, e.g. by transcription and/or reverse transcription.

In one embodiment, the REST-silencing sequence is a RNA interference molecule as is well known in the art. The sequence may be an antisense sequence. Examples of suitable sequences include double-stranded RNA (dsRNA) molecules or analogues thereof, double-stranded DNA (dsDNA) molecules or analogues thereof, short hairpin RNA (shRNA) molecules, small interfering RNA (siRNA) molecules and antisense oligonucleotides. microRNA molecules may also be used.

In a preferred embodiment, the REST-silencing sequence is a shRNA molecule. Examples of shRNA molecules that can be used to silence REST expression include the following sequence (SEQ ID NO: 4) shown here in its DNA form:

gggcctatttcccatgattccttcatatttgcatatacgatacaaggctgt
tagagagataattggaattaatttgactgtaaacacaaagatattagtaca
aaatacgtgacgtagaaagtaataatttcttgggtagtttgcagttttaaa
attatgttttaaaatggactatcatatgcttaccgtaacttgaaagtattt
cgatttcttggctttatatatcttgtggaaaggacgaaacaccggtACTTC
AATAAGGACTTGCTCCACACGTGAGGAGCAAGTCCTTATTGAAGTTTTTTG
AATTCTCGACCggatccCGGCCGCCCCCTTCACCGAgggcctatttcccat
gattccttcatatttgcatatacgatacaaggctgttagagagataattgg
aattaatttgactgtaaacacaaagatattagtacaaaatacgtgacgtag
aaagtaataatttcttgggtagtttgcagttttaaaattatgttttaaaat
ggactatcatatgcttaccgtaacttgaaagtatttcgatttcttggcttt
atatatcttgtggaaaggacgaaacaccggtAGTACACATTAACCAAATGG
CACACGTGAGCCATTTGGTTAATGTGTACTTTTTTgaattctcgacctcga
gaagcttgatatcgaattc

General methods for identifying suitable siRNA, microRNA and antisense oligonucleotide molecules for RNA interference are well known in the art.

Since the nucleic acid sequence of REST is known (eg human REST has the sequence under NCBI Accession No NG_029447), the skilled person would readily appreciate how to design and test other candidate RNAi molecules (eg shRNA molecules) targeted to REST.

By way of example, antisense oligonucleotides typically are about 5 nucleotides to about 30 nucleotides in length, about 10 to about 25 nucleotides in length, or about 20 to about 25 nucleotides in length. For a general discussion of antisense technology, see, e.g., Antisense DNA and RNA, (Cold Spring Harbor Laboratory, D. Melton, ed., 1988).

By way of further example, the sense strand of an siRNA is typically about 20-24 nucleotides in length and the complementary sense and antisense regions of shRNAs are also typically about 20-24 nucleotides. For general information on siRNA technology, see, e.g. siRNA Design: Methods and Protocols, (Methods Mol Biol, vol 942, D. J. Taxman, ed., 2013). For general information on shRNA technology, see, e.g. Moore et al (2010) Methods Mol Biol 629:141-158.

Appropriate chemical modifications of the antisense oligonucleotide inhibitors of the present disclosure can be made to ensure stability of the antisense oligonucleotides as is commonplace in the art. Changes in the nucleotide sequence and/or in the length of the antisense oligonucleotide can be made to ensure maximum efficiency and thermodynamic stability of the inhibitor. Again, such sequence and/or length modifications are readily determined by one of ordinary skill in the art.

Although RNAi molecules can contain nucleotide sequences that are fully complementary to a portion of the target nucleic acid, it will be appreciated that 100% sequence complementarity between the RNAi probe and the target nucleic acid is not required.

RNAi molecules can be synthesized by standard methods known in the art, e.g., by use of an automated synthesizer. RNAs produced by such methodologies tend to be highly pure and to anneal efficiently to form siRNA duplexes or shRNA hairpin stem-loop structures. Following chemical synthesis, single stranded RNA molecules are deprotected, annealed to form siRNAs or shRNAs, and purified (e.g., by gel electrophoresis or HPLC). Alternatively, standard procedures may be used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donze and Picard, Nucleic Acids Res. 2002; 30:e46; and Yu et al, Proc. Natl. Acad. Sci. USA 2002; 99:6047-6052). Similarly, an efficient in vitro protocol for preparation of shRNAs using T7 RNA polymerase has been described (Yu et al, supra). The sense and antisense transcripts may be synthesized in two independent reactions and annealed later, or may be synthesized simultaneously in a single reaction.

It will be appreciated that the gene expression system may comprise more than one nucleotide sequence encoding a neuronal conversion factor. In this way, multiple neuronal conversion factors can be introduced into a cell. The gene expression system may comprise 2 or more, 3 or more, 4 or more, or 5 or more nucleotide sequence encoding a neuronal conversion factors. In a particularly preferred embodiment, the gene expression system comprises at least two nucleotide sequences encoding the respective neuronal conversion factors ASCL1 and BRN2.

It will be appreciated that the gene expression system may comprise more than one REST-silencing sequence, such as 2 or more, 3 or more, 4 or more, or 5 or more REST-silencing sequences. In a particularly preferred embodiment, the gene expression system comprises two REST-silencing sequences (eg shRNA molecules).

It will be appreciated that one or more or all of the nucleotide sequences of the gene expression system of the first aspect of the invention (eg of components (a) and (b)) may be incorporated into a vector.

In one embodiment, the nucleotide sequences of (a) (e.g. the nucleotide sequences of (a) (i) and (a) (ii)) are comprised in a single vector.

In a further embodiment, the nucleotide sequences of (a) and (b) (e.g. the nucleotide sequences of (a) (i), (a) (ii) and (b)) are comprised in a single vector.

By vector it will be understood that we include the meaning of a vehicle which is able to artificially carry foreign (i.e. exogenous) genetic material into a cell (e.g. a prokaryotic (eg bacterial) or eukaryotic (eg mammalian) cell) where it can be replicated and/or expressed. Examples of vectors include non-mammalian nucleic acid vectors, such as bacterial artificial chromosomes (BACs), yeast artificial chromosomes (YACs), P1-derived artificial chromosomes (PACs), cosmids or fosmids. Other examples of vectors include viral vectors such as retroviral vectors and lentiviral vectors.

The precise polynucleotide sequence of the vector will depend upon the nature of the intended host cell, the manner of the introduction of the polynucleotide of the first aspect of the invention into the host cell, and whether episomal maintenance or integration is desired. The vector may comprise at least one selectable marker such as antibiotic resistance (e.g. kanamycin or neomycin). However, it will be appreciated that viral vectors, e.g. lentiviral vectors, do not typically comprise selectable markers in the nucleic acid molecule that is packaged into viral particles.

In a particularly preferred embodiment, the vector is a lentiviral vector which are well known in the art.

Lentiviral vectors, such as those based upon Human Immunodeficiency Virus Type 1 (HIV) are widely used as they are able to integrate into non-proliferating cells. Viral vectors can be made replication defective by splitting the viral genome into separate parts, e.g., by placing on separate plasmids. For example, the so-called first generation of lentiviral vectors, developed by the Salk Institute for Biological Studies, was built as a three-plasmid expression system consisting of a packaging expression cassette, the envelope expression cassette and the vector expression cassette. The “packaging plasmid” contains the entire gag-pol sequences, the regulatory (tat and rev) and the accessory (vif, vpr, vpu, net) sequences. The “envelope plasmid” holds the Vesicular stomatitis virus glycoprotein (VSVg) in substitution for the native HIV-1 envelope protein, under the control of a cytomegalovirus (CMV) promoter. The third plasmid (the “transfer plasmid”) carries the Long Terminal Repeats (LTRs), encapsulation sequence (ψ), the Rev Response Element (RRE) sequence and the CMV promoter to express the transgene inside the host cell.

The second lentiviral vector generation was characterized by the deletion of the virulence sequences vpr, vif, vpu and nef. The packaging vector was reduced to gag, pol, tat and rev genes, therefore increasing the safety of the system.

To improve the lentiviral system, third-generation vectors have been designed by removing the tat gene from the packaging construct and inactivating the LTR from the vector cassette, therefore reducing problems related to insertional mutagenesis effects.

In a particularly preferred embodiment, therefore, the gene expression system is comprised within a third generation lentiviral vector

The various lentivirus generations are described in the following references: First generation: Naldini et al. (1996) Science 272(5259): 263-7; Second generation: Zufferey et al. (1997) Nat. Biotechnol. 15(9): 871-5; Third generation: Dull et al. (1998) J. Virol. 72(11): 8463-7, all of which are incorporated herein by reference in their entirety. A review on the development of lentiviral vectors can be found in Sakuma et al. (2012) Biochem. J. 443(3): 603-18 and Picanço-Castro et al. (2008) Exp. Opin. Therap. Patents 18(5):525-539.

Where the gene expression system encodes more than one protein, it will be appreciated that it may be configured such that the coding sequences for the proteins are transcribed either as separate transcripts, for example under the control of separate promoters (i.e. each coding sequence being transcribed into a distinct mRNA molecule that is translated into one protein) or are transcribed as a single transcript, for example under the control of one promoter (i.e. the mRNA molecule is multicistronic and contains more than one coding region which are translated into the different proteins; the multiple coding regions may be separated by an internal ribosome entry sequence (IRES)). For example, when the gene expression system encodes ASCL1 and BRN2, it will be understood that ASCL1 and BRN2 may be translated from distinct mRNA molecules, each transcribed independently, or that ASCL1 and BRN2 may be translated from one mRNA (e.g. one that is bicistronic, having two coding regions). Molecular biological methods for cloning and engineering genes and cDNAs, including their expression as multiple or single transcripts, are well known in the art, as exemplified in “Molecular cloning, a laboratory manual”, third edition, Sambrook, J. & Russell, D. W. (eds), Cold Spring Harbor Laboratory Press, Cold Spring Harbor, N.Y., incorporated herein by reference. Preferably, the proteins encoded by the gene expression system are transcribed independently.

The vectors of the invention typically include heterologous control sequences, including, but not limited to, constitutive promoters, tissue or cell type specific promoters, regulatable or inducible promoters, enhancers, and the like.

Exemplary promoters include, but are not limited to: the phosphoglycerate kinase-1 (PGK) promoter, the cytomegalovirus (CMV) immediate early promoter, the RSV LTR, the MoMLV LTR, a simian virus 40 (SV40) promoter and a CK6 promoter, a transthyretin promoter (TTR), a TK promoter, a tetracycline responsive promoter (TRE), an HBV promoter, and an hAAT promoter. The nucleotide sequence of these and numerous additional promoters are known in the art. The relevant sequences may be readily obtained from public databases and incorporated into vectors for use in practicing the present invention.

In an embodiment, the nucleotide sequences are under the control of a constitutive promoter such as a PGK promoter or under the control of a regulatable promoter such as a doxycycline regulatable promoter.

In a particularly preferred embodiment, the gene expression system comprises a nucleotide sequence that encodes ASCL1, and a nucleotide sequence that encodes BRN1, wherein the respective nucleotide sequences are under the control of respective promoters. It will be appreciated that the expression of ASCL1 and BRN2 may be under the control of the same or different promoters. Preferably, their expression is under the control of the same promoter, and most preferably under the control of the PGK promoter.

It will be understood that the nucleotide sequences encoding ASCL1 and BRN2 may be incorporated into the gene expression system (e.g. vector such as a lentiviral vector) in any order. For example, the nucleotide sequence encoding ASCL1 may be at the 5′ end of the nucleotide sequence encoding BRN2, or the nucleotide sequence encoding BRN2 may be at the 5′ end of the nucleotide sequence encoding ASCL1. The inventors have found that placing the nucleotide sequence encoding BRN2 to the 5′ end of the nucleotide sequence encoding ASCL1 results in the highest yield of induced neurons. Hence, it is preferred if the nucleotide sequences are configured in the order pB.pA, i.e. the nucleotide sequence encoding BRN2 (“B”) under the control of a promoter (p), is positioned at the 5′ end of the nucleotide sequence encoding ASCL1 (“A”) under the control of a promoter (p). Preferably, the promoters are the constitutive promoter pGK, and so it will be appreciated that the gene expression system may comprise nucleotide sequences configured in the order pGK.B.pGK.A.

In a further embodiment, the gene expression system further comprises one or more (e.g. 2 or more, 3 or more, 4 or more, or 5 or more) enhancer sequences such as the Woodchuck Heptatitis Virus Posttranscriptional Regulatory Element (WPRE). This is a DNA sequence that when transcribed, creates a tertiary structure enhancing expression and is commonly used in molecular biology to increase expression of genes in viral vectors (See Donello, J E; Loeb, J E; Hope, T J (Jun 1998). “Woodchuck hepatitis virus contains a tripartite posttranscriptional regulatory element”. J. Virol. 72 (6): 5085-92. PMC 110072. PMID 9573279). The sequence of this element is well known in the art and would be easily incorporated into the gene expression system of the invention by the skilled person as a matter of routine.

Again, it will be appreciated that the one of more enhancer sequence(s) may be positioned at different positions and/or distances to the nucleotide sequences encoding the neuronal conversion factors (e.g. nucleotide sequences encoding ASCL1 and BRN2).

The invention also provides a gene expression system comprising

a. (i) a nucleotide sequence encoding ASCL1;
(ii) a nucleotide sequence encoding BRN2; and

b. a molecule capable of inhibiting REST.

By a molecule capable of inhibiting REST we include the meaning of any molecule (e.g. a small molecule) that reduces at least one function of REST. By reduces at least one function of REST, we include the meaning of reducing at least one function of REST to less than 90% of the at least one function of REST apparent in the absence of the molecule, such as less than 80%, 70%, or 60%, and preferably less than 50%, 40%, 30%, 20% or 10% of the at least one function of REST apparent in the absence of the molecule, and most preferably to an undetectable level of the at least one function of REST. Any suitable method of determining the at least one function of REST can be used as is known in the art, such as by luciferase assay (e.g. as described in Charbord et al, 2013, Stem Cells, 31(9):1816-1828, the entire contents of which are incorporated herein by reference). Suitable molecules capable of inhibiting REST include REST Inhibitor, X5050 (Calbiochem, Merck catalogue number 506026). We consider that it would be a matter of routine for the skilled person to identify further such molecules, e.g. using the methods described in Charbord et al, 2013, supra.

It will be appreciated that by inhibiting REST we also include the meaning of reducing the amount of REST, suppressing REST-expression, and/or reducing the level or transcription and/or translation of REST, as described above. Hence, a molecule capable of inhibiting REST includes a REST-silencing sequence capable of suppressing REST-expression as described above, such as a RNA interference molecule.

A second aspect of the invention provides a cell comprising the gene expression system of the first aspect of the invention. Such a cell may be one into which the gene expression system of the first aspect of the invention is introduced so that it can be reprogrammed to be an induced neuron cell as described further below.

Preferences for the gene expression system include those described above in relation to the first aspect of the invention.

The cell can be prokaryotic or eukaryotic.

It is appreciated that construction and amplification of the gene expression system of the first aspect of the invention is conveniently performed in bacterial cells (e.g. when the gene expression system is in the form of a bacterial plasmid, BAC, PAC, cosmid, fosmid etc.), in yeast cells (e.g. when the gene expression system is in the form of a YAC), and in mammalian cells (e.g. when the gene expression system is comprised within a viral vector, typically encapsulation of nucleic acid in a viral particle) whereas the use of the gene expression system for neuronal conversion is typically limited to mammalian cells.

It will be appreciated that the gene expression system can be introduced into a cell by any of the known techniques, namely transformation, transduction or transfection, all of which are standard techniques in the art. Preferably, the gene expression system is comprised within a viral vector and the gene expression system is therefore introduced into the cell by transduction.

It is preferred if the cell is a mammalian cell such as any vertebrate cell including a cell from a human, a mouse, a rat, or a monkey.

The cell may be a primary cell, a secondary cell or a cell line.

In one embodiment, the cell is a primary cell that has been cultured from a mature cell type, for example the cell may be a primary fibroblast.

Thus, it will be appreciated that the cell may be derived from a biopsy sample obtained from an animal such as a human. Preferably, the biopsy sample is one that comprises fibroblasts, such as a skin punch biopsy or a lung biopsy. The preparation of primary cells from biopsy samples is routine in the art and any suitable technique can be used, including those described in the Examples.

As mentioned above, the gene expression system of the invention has value in modelling neurodegenerative diseases, and so it is particularly desirable if the biopsy sample is obtained from an individual with a neurodegenerative disorder. For example, the biopsy sample may be obtained from an individual with a familial or sporadic form of Alzheimer's disease or a familial or sporadic form of Parkinson's disease. Similarly, the biopsy sample may be obtained from an individual with Huntington's disease. However, it is also appreciated that the biopsy sample may be obtained from a healthy individual, which may serve as a useful control in disease modelling or drug screening experiments.

Alternatively, the cell of the second aspect of the invention may be one that is derived from a cell line. For example, the cell may be a fibroblast derived from the human fetal lung fibroblast (HFL1) cell line (ATCC-CCL-153). Cell lines are a convenient source of cells to use in the construction, development and testing of the gene expression system of the invention.

Following introduction of the gene expression system of the invention into a cell, culturing of the cell results in the conversion of the cell into an induced neuron. Thus, also included in the cell of the second aspect of the invention is a cell into which the gene expression system of the invention has been introduced and which has been cultured until converted into an induced neuron directly (e.g. without transitioning via a proliferative stem cell intermediate). Like neurons, induced neurons are unable to undergo mitosis and so can be considered also to be in a post-mitotic state, that is they are post-mitotic.

As demonstrated in the Examples, the inventors have shown that the reprogramming efficiency of the gene expression system is not affected by the passage number of the starting primary culture (eg primary fibroblast cell culture), which lends the technology particularly well to large scale disease modelling. Thus, it will be appreciated that the cell may have been passaged numerous times before the gene expression system is introduced, in which case the cell is a secondary cell. For example, the cell may have been passaged at least 1, 2, 3, 4, 5, 6, 7, 8, 9, 10, 15, 20, 25, 30, 35, 40, 45 or 50 times prior to introduction of the gene expression system of the invention. In an embodiment, the cell is one that was passaged at least 3 times before the gene expression system is introduced. In a further embodiment, the cell is one that was not passaged more than 50 times before the gene expression system is introduced.

A third aspect of the invention provides a method of inducing neurons directly from fibroblast cells comprising the step of introducing the gene expression system of the first aspect of the invention into a fibroblast cell. The method is typically in vitro or ex vivo.

Preferences for the gene expression system and fibroblast cell include those described above in relation to the first and second aspects of the invention.

Although the method is described in the context of reprogramming fibroblast cells into induced neurons, it will be appreciated that other somatic cells may be similarly reprogrammed. Hence, wherever the third aspect of the invention is described in relation to fibroblast cells, it will be understood that any other somatic cell could be used, e.g. a blood-derived cell.

The gene expression system can be introduced into the cell using any appropriate technique known in the art, such as transformation, transduction and transfection.

In a preferred embodiment, the gene expression system is comprised in a viral vector (e.g. lentiviral vector), and the viral vector is introduced into the fibroblast by transduction as described in the Examples.

Generally, the fibroblast cells will be cultured in a growth medium suitable for growth of fibroblast cells (i.e. fibroblast medium) before and during the process of introducing the gene expression system into the cell. Such media is well known in the art and are commercially available from multiple suppliers. One example is Dulbecco's Modified Eagle Medium (DMEM)+Glutamax (Gibco) with 100 mg/mL penicillin/streptomycin (Sigma), and 10% FBS (Biosera) as used in the Examples.

To assist differentiation into neuron cells, in one embodiment, following introduction of the gene expression system into a fibroblast cell, the cells are cultured in a neural differentiation medium, e.g. a cell culture medium suitable for neural differentiation. The neural differentiation medium is preferably a serum free medium. Any medium that supports culturing of induced neurons may be used. The neural differentiation medium preferably comprises basal medium and hormone supplement, as are well known in the art.

Preferred examples for neural differentiation media are cell culture media containing supplements selected from the group consisting of N2, B27, N2B27 and/or G5 supplement. A variety of such media are commercially available, one suitable example being NDiff227 which is commercially available from Takara-Clontech (see Ying et al, 2003, Nat Biotechnol 21(2):183-186 for the original formulation).

Typically, the neural differentiation medium is supplemented with one or more growth factors, such as any of LM-22A4, GDNF, NT3 and db-cAMP and/or the neural differentiation medium is supplemented with one or more small molecules, such as any of CHIR99021, SB-431542, noggin, LDN-193189 and valproic acid sodium salt.

In a particularly preferred embodiment, the fibroblast cells are cultured in NDiff227 medium supplemented with growth factors at the following optional concentrations: LM-22A4 (2 μM, R&D Systems), GDNF (2 ng/ml, R&D Systems), NT3 (10 ng/μl, R&D Systems) and db-cAMP (0.5 mM, Sigma); and with small molecules at the following optional concentrations CHIR99021 (2 μM, Axon), SB-431542 (10 μM, Axon), noggin (0.5 μg/ml, R&D Systems), LDN-193189 (0.5 μM, Axon), and valproic acid sodium salt (VPA; 1 mM, Merck Millipore)

Cell culture techniques are standard in the art and any suitable protocol can be used. Generally, the cells are cultured for 10-125 days, for example for 25 days, until they form induced neuron cells.

Conveniently, the cells are cultured on an immobilised support such as in a multi-well plate format. It may be desirable to replete the cells onto a fresh support during the culture process, for example after 10-12 days. The fresh supports may be ones particularly suited to neuron cell culture such as ones coated with any one or more of polyornithine, fibronectin and laminin or similar.

At least some of the neuronal differentiation medium may be replaced at regular intervals (eg 2-4 days; typical minimum is every 4 days).

In an embodiment, the method further comprises assessing the cell for one or more neuronal characteristics including morphological properties (for example neurite outgrowth, the presence of a soma/cell body, dendrites, axon and/or synapses); expression of neuronal specific markers such as MAP2, NF-H, bIII-tubulin, NeuN, Synapsin and Tau; excitatory or inhibitory membrane properties, for example as evidence by expression of vGlut and/or Gad67; and membrane depolarization capacity. Methods for assessing neuronal characteristics are well known in the art and include all of those described in relation to the first aspect of the invention. For example, any of immunocytochemistry, fluorescence activated cell sorting, and electrophysiology techniques may be used. These techniques and other suitable techniques are described in the examples.

A fourth aspect of the invention provides an induced neuron cell obtainable by carrying out the method of the third aspect of the invention.

Preferences for the cell include those described in relation to the second aspect of the invention. The cell may be one that was passaged at least 3 times, or was passaged up to 50 times before introduction of the gene expression system.

A fifth aspect of the invention provides the use of a gene expression system according to the first aspect of the invention or a cell according to the second or fourth aspects of the invention in disease modelling, or in diagnostics or in drug screening.

A sixth aspect of the invention provides a gene expression system according to the first aspect of the invention or a cell according to the second or fourth aspects of the invention for use in medicine.

For example, the invention includes a gene expression system according to the first aspect of the invention or a cell according to the second or fourth aspects of the invention for use in diagnostics, or in cell therapy or in gene therapy. For example, the gene expression system according to the first aspect of the invention or cell according to the second or fourth aspect of the invention may be used in the preparation of cells or tissue for gene or cell therapy.

The invention also includes a pharmaceutical composition comprising a gene expression system according to the first aspect of the invention or a cell according to the second aspect of the invention, and a pharmaceutically acceptable carrier.

Whilst it is possible for the agent of the invention (i.e. gene expression system or cell) to be administered alone, it is preferable to present it as a pharmaceutical formulation, together with one or more acceptable carriers. The carrier(s) must be “acceptable” in the sense of being compatible with the therapeutic agent and not deleterious to the recipients thereof. Typically, the carriers will be water or saline which will be sterile and pyrogen free.

Where appropriate, the formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above the formulations of this invention may include other agents conventional in the art having regard to the type of formulation in question.

The amount of the agent which is administered to the individual is an amount effective to combat the particular individual's condition. The amount may be determined by the physician.

Preferably, in the context of any aspect of the invention described herein, the subject or individual is a human. Alternatively, the subject may be an animal, for example a domesticated animal (for example a dog or cat), laboratory animal (for example laboratory rodent, for example mouse, rat or rabbit) or an animal important in agriculture (i.e. livestock), for example horses, cattle, sheep or goats.

A seventh aspect of the invention provides a method of screening for a compound that alters at least one disease related biomarker, the method comprising

a. exposing an induced neuron according to the second or fourth aspect of the invention to at least one chemical compound to be tested

b. registering the level of at least one disease related biomarker

c. comparing the registered level of at least one disease related biomarker in b. with one or more reference levels; and

d. selecting at least one compound that alters the level of at least one disease related biomarker with the one or more reference levels.

The chemical compound may be any compound including any of an antibody, a peptide, a peptidomimetic, a natural product, a carbohydrate, an aptamer, or a small organic molecule or synthetic molecule.

In an embodiment, the induced neuron is exposed to more than one chemical compound in step (a). This may be desirable where it is known or believed that the more than one chemical compounds are only effective in combination, rather than when alone.

As is well known in the art, a biomarker can be any characteristic that can be objectively measured and evaluated to provide an indication of a normal biological process, a pathological process and/or a pharmacological response to a therapeutic intervention. By a “disease-related biomarker”, we include the meaning of any biomarker, the assessment of which can be used to provide an indication of disease status. For example, a disease-related biomarker may provide an indication of the probable effect of treatment on a subject (a risk indicator or predictive biomarker), it may provide an indication as to whether a disease already exists (a diagnostic biomarker), or it may provide an indication on how such a disease may develop in an individual case regardless of the type of treatment (a prognostic biomarker).

In one embodiment, the biomarker is a molecule such as a nucleic acid, a protein or a metabolite, whose concentration reflects the severity or presence of some disease state. For example, the disease related biomarker may be a molecule that is not normally present or detectable in a healthy cell or tissue, but which is present and detectable in a disease cell or tissue, or it may be a molecule that is present at a different concentration in a disease cell or tissue to the concentration of the molecule in a healthy cell or tissue. Detection and quantification of molecules such as nucleic acids, proteins and metabolites can be carried out routinely using standard methods in the art. For example, nucleic acids can be detected using PCR or rRT-PCR, proteins can be detected using ELISA and antibody binding assays, and metabolites can be measured by known analytical chemistry techniques including HPLC, LC and/or mass spectrometry.

It will be appreciated that the disease related biomarker may or may not be an intracellular molecule, and so the term includes both extracellular and/or intracellular molecules.

In an alternative embodiment, the biomarker is not a molecule but is another otherwise detectable characteristic such as a detectable activity or function or a detectable change in cell morphology or any other phenotype. Again, the skilled person would be readily able to assess the activity or function or morphology of, for example, neuronal cells making use of standard practices in the art including patch-clamp technology, imaging and microscopy.

The induced neuron cells produced by the technology of the present invention are believed to be especially useful in the study of neurodegenerative diseases and treatments thereof, and so in a preferred embodiment, the disease related biomarker is a biomarker of a neurological disorder, such as any of Alzheimer's disease, Parkinson's disease or Huntington's disease.

Such disease related biomarkers are well known to the skilled person and can be readily identified by interrogating the scientific literature. Indeed, as research efforts continue to document disease related biomarkers for more and more diseases, systems are being put in place to extract them efficiently (see, for example, Bravo et al “A Knowledge-Driven Approach to Extract Disease-Related Biomarkers from the Literature” BioMed Research International, Volume 2014 (2014), Article ID 253128, 11 pages).

In one embodiment, at least 2, 3, 4, 5, 6, 7, 8, 9 or 10 or more disease related biomarkers are assessed in step (b). Assessing more than one disease related biomarker is often desirable where the disease is one that has several disease related biomarkers, and finding a compound that alters the level of some or all of them may improve the chances of finding a therapy. However, it will be appreciated that the method may only require the assessment of one disease related biomarker.

By “registering the level of” or “registering the measured level of” the disease related biomarker in step (b), we include the meaning of noting the level of disease related biomarker that is apparent following exposure of the induced neuron to the chemical compound. This may involve measuring the level of the disease related biomarker, or noting an already measured level of the disease related biomarker. The noted or registered level can then be compared with a reference level of the disease related biomarker.

By “reference level of the disease related biomarker” in step (c), we include the meaning of a level of the disease related biomarker that can be compared to the level registered in step (d) in order to determine whether there has been an alteration in the level of the disease related biomarker caused by the chemical compound. The reference level may be one that was registered in the same or different cell immediately exposure to the chemical compound, or one that was registered in advance of the experiment and stored for comparative use.

If the level(s) of the one or more disease related biomarkers registered in step (b) is different from the one or more reference levels in step (c), then the chemical compound is one that alters the one or more disease-related biomarker.

It is appreciated that the identification of a chemical compound that alters a disease related biomarker may be an initial step in a drug screening pathway, and the identified agents may be further selected e.g. for efficacy in a model of the disease in question. Thus, the method may further comprise the step of testing the chemical compound in a model (eg animal model) of the disease in question (eg neurodegenerative disease).

It is appreciated that these methods may be a drug screening methods, a term well known to those skilled in the art, and the chemical compound may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 Daltons and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

In an embodiment, the identified chemical compound is modified, and the modified compound is tested for the ability to alter one or more disease related biomarkers.

Compounds may also be subjected to other tests, for example toxicology or metabolism tests, as is well known to those skilled in the art.

An eighth aspect of the invention provides a method for detecting the presence, progression or early stage onset/development of an age related neurological clinical condition in an individual comprising

a. introducing the gene expression system of the first aspect of the invention into fibroblasts in a biopsy sample obtained from the individual;

b. registering the level of at least one potential disease-associated phenotype or biomarker in these cells at the stage of induced neuron

c. comparing the registered level of at least one potential disease-associated phenotype or biomarker in b. with one or more reference levels; and

d. stratifying the sample based on the correlation to the reference levels in c. as indicative of the absence, the presence, progression or early stage onset/development of an age related neurological clinical condition.

Preferences for the gene expression system, biopsy sample, and methods of introduction of gene expression system into fibroblasts (eg transduction) include those described above in relation to the first and second aspects of the invention.

Conveniently, the biopsy sample is cultured to expand the fibroblast cell population, for example by culturing the cells in fibroblast growth medium as described above and in the Examples, before the gene expression system is introduced.

In an embodiment, the age related neurological clinical condition is selected from the group comprising Familial and sporadic Alzheimer's disease; Familial and sporadic Parkinson's disease; and Huntington's disease.

For the avoidance of doubt, the terms “disease associated” and “disease related” are equivalent herein, and the term “disease-associated phenotype” can be considered the same as “disease related biomarker”.

By a potential disease-associated phenotype or biomarker we include the meaning of any measurable biomarker that has the potential to be a disease related biomarker, eg a disease related biomarker of a neurological condition such as any of Alzheimer's disease, Parkinson's disease or Huntington's disease.

For example step (b) may involve measuring the level of an intracellular protein in the induced neuron which is not currently known to be a disease related biomarker, but once its registered level is compared with one or more reference levels in step (c), for example as measured in induced neurons obtained from an individual having a known status of the absence, presence, progression or early stage onset/development of the age related neurological condition, it may be possible to correlate the level of the intracellular protein with the status of the absence, presence, progression or early stage onset/development of the age related neurological condition. Such correlation will be possible assuming that the level of the intracellular protein does indeed vary according to the status of the absence, presence, progression or early stage onset/development of the age related neurological condition. If the level of the intracellular protein does not so vary then it cannot be considered a disease related biomarker.

By stratifying the sample based on the correlation to the reference levels in (c), we include the meaning of attributing the sample to an individual with a particular status of the absence, presence, progression or early stage onset/development of the age related neurological condition. For example, if the level of a given intracellular protein varies according to the absence, presence, progression or early stage onset/development of the age related neurological disorder, then it will be possible to attribute any given sample in which the level of that intracellular protein has been determined to the correct status of that age related neurological disorder.

A ninth aspect of the invention includes the use of a REST inhibitor, e.g. a REST-silencing sequence capable of suppressing REST-expression, in directly converting a fibroblast into an induced neuron.

The invention also provides a method of directly converting a fibroblast into an induced neuron comprising contacting the fibroblast with a REST inhibitor, e.g. a REST-silencing sequence capable of suppressing REST-expression. The method may be performed in vitro or ex vivo.

Preferences for the REST inhibitor, e.g. a REST-silencing sequence capable of suppressing REST-expression, and fibroblast are described above in relation to the first and second aspects of the invention.

It is preferred if the REST-silencing sequence is used in combination with one or more known neuronal conversion factors as described above in relation to the first aspect of the invention, such as ASCL1 and BRN2.

The invention also provides a kit of parts for inducing neurons in an animal fibroblast cell, such as a human fibroblast cell, comprising a gene expression system as described above in relation to the first aspect of the invention, in particular to a gene expression system comprising a first nucleotide sequence encoding a peptide of Ascl1, a second nucleotide sequence encoding a peptide of Brn2 and a third nucleotide sequence of at least one nucleotide sequence encoding a REST-silencing sequence, such as short hairpin REST sequences suppressing REST-expression.

The listing or discussion of an apparently prior-published document in this specification should not necessarily be taken as an acknowledgement that the document is part of the state of the art or is common general knowledge.

The invention will now be described with reference to the following Figures and Examples.

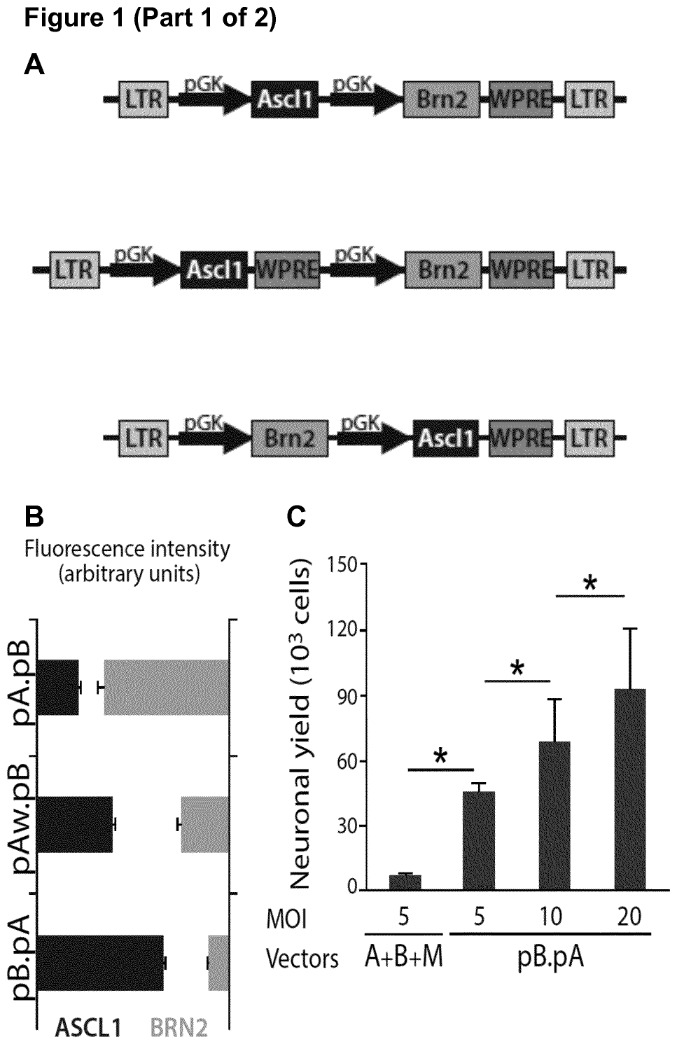

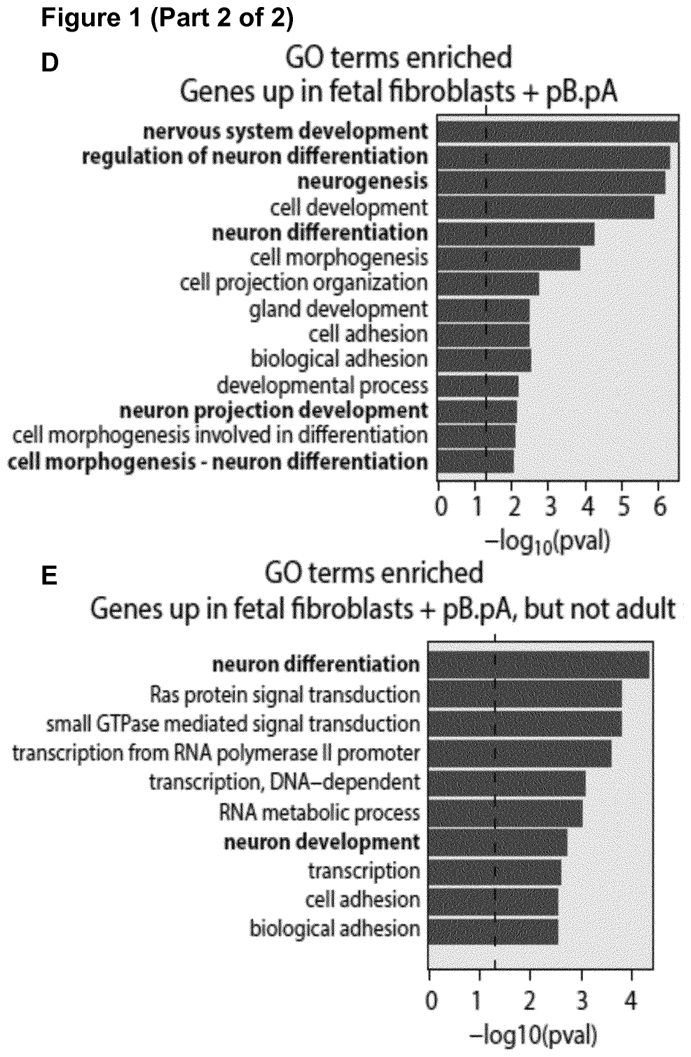

FIG. 1. Bicistronic approach successfully reprograms fetal fibroblasts but fails to reprogram adult fibroblasts.

(A) Vector maps of constructs containing the neural conversion factors ASCL1 coding for MASH1 and BRN2A as well as woodchuck hepatitis post-transcriptional element (WPRE) at different positions. (B) Quantitative analysis showing the difference in fluorescence intensity of ASCL1 (red bar graphs) and BRN2a (yellow bar graphs) following transduction with the different constructs.

(C) Quantification of the number of iNs converted 12 days after transduction with either Pgk.Ascl1+Pgk.Brn2a+Pgk.Myt1L or pB.pA. Data are expressed as means±SEM. * p<0.05. (D) Gene ontology enrichment analysis reveal significant enrichment of neuronal genes (in bold) among the up-regulated genes in the pB.pA transduced fetal fibroblasts. (E) Gene ontology enrichment analysis showing the genes associated with neurons (in bold) that are up-regulated in the pB.pA transduced fetal fibroblasts but not in the adult fibroblasts transduced with pB.pA.

Data information: Data are expressed as mean±SEM and are from biological replicates (n=3). *p<0.05.

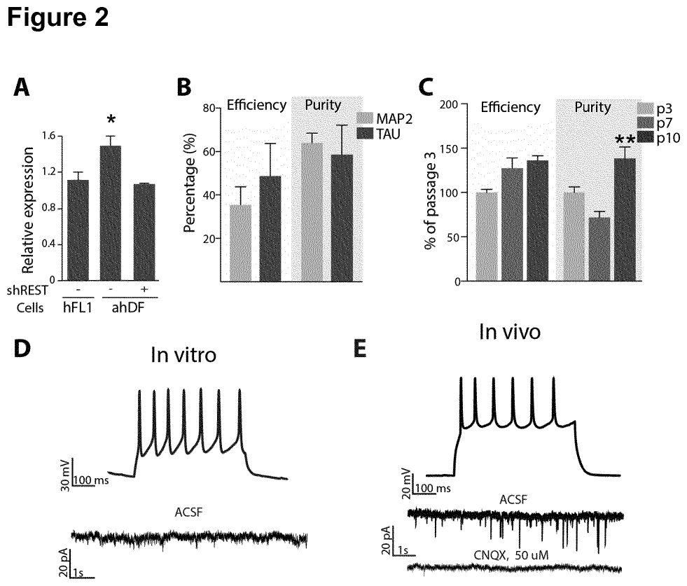

FIG. 2. REST Knockdown promotes the pB.pA-driven reprogramming of adult human fibroblasts.

(A) qPCR analysis of REST gene expression. (B) Quantification of neuronal efficiency and purity of pB.pA+RESTi reprogrammed adult human dermal fibroblasts from five healthy donors (61-71 years old). (C) Quantification of neuronal efficiency and purity of an adult human dermal fibroblast line reprogrammed with pB.pA+RESTi at different passages. (D) In vitro Patch clamp recordings of adult iNs depicting repetitive current-induced action potentials indicative of mature neuronal physiology at 12-15 weeks post transduction. (E) Presence of repetitive current-induced action potentials and spontaneous post-synaptic currents in vivo 8 weeks following transplantation.

Data information: Abbreviations: ahDF: adult human dermal fibroblasts; shREST: short hairpin RNA against REST. Data are expressed as mean±SEM and are from biological replicates (n=3-4). *p<0.05.

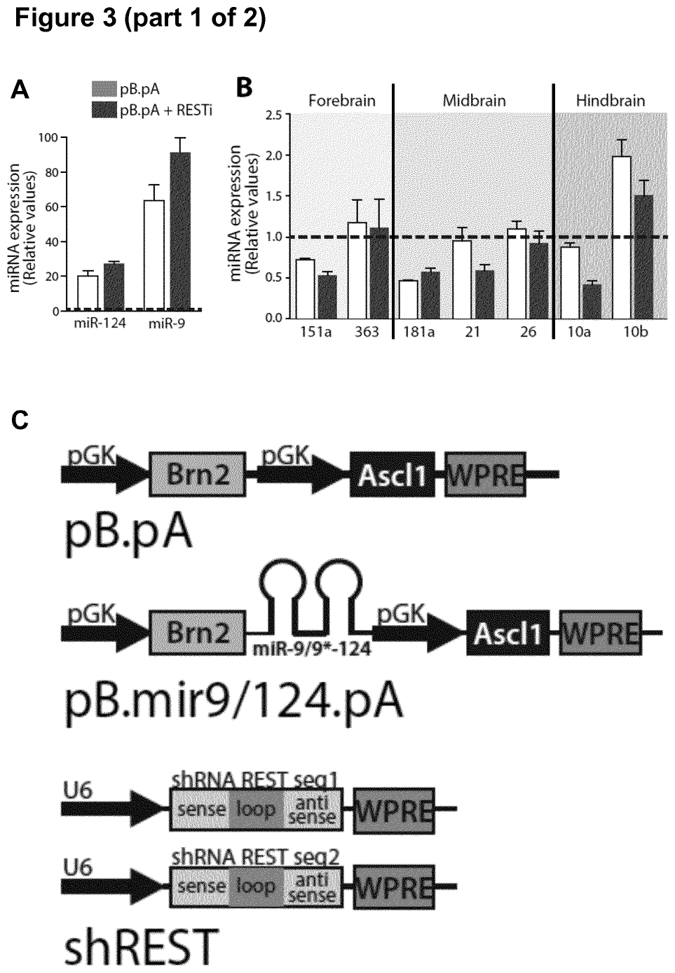

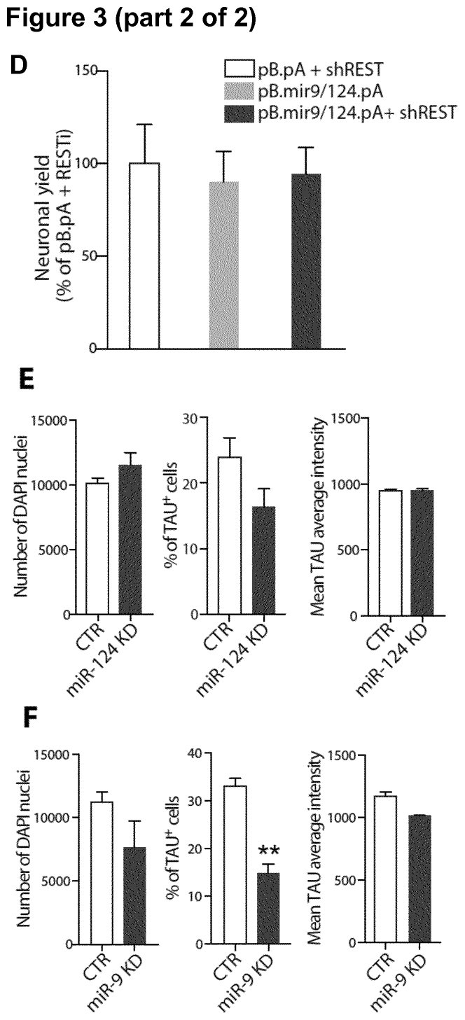

FIG. 3. Neuronal microRNA expression partly drives neuronal reprogramming of adult fibroblasts.

(A) qPCR measurements of miR-124 and miR-9 in adult fibroblasts reprogrammed with pB.pA only or pB.pA+RESTi and normalized on the non-transduced fibroblast values (yellow dashed line). (B) Region-specific microRNAs qPCR measurements in adult fibroblasts reprogrammed with pB.pA only or pB.pA+RESTi and normalized on the non-transduced fibroblast values (yellow dashed line). (C) Vector maps of constructs containing the transcription factors Ascl1 and Brn2a with and without miR-9 and miR-124, as well as the shRNA sequences against REST. (D) Quantification of the neuronal yield as assessed by MAP2 expression in adult fibroblasts transduced with different reprogramming vectors. (E) Quantification of the total number of cells as well as the percentage of TAU⁺ cells and the average fluorescence intensity in adult iNs with and without miR-124. (F) Quantification of the total number of cells as well as the percentage of TAU⁺ cells and the average fluorescence intensity in adult iNs with and without miR-9 knockdown.

Data information: Abbreviations: CTR: Control; KD: Knockdown. Data are expressed as mean±SEM and are from biological replicates (n=3-4). *p<0.05, **p<0.01.

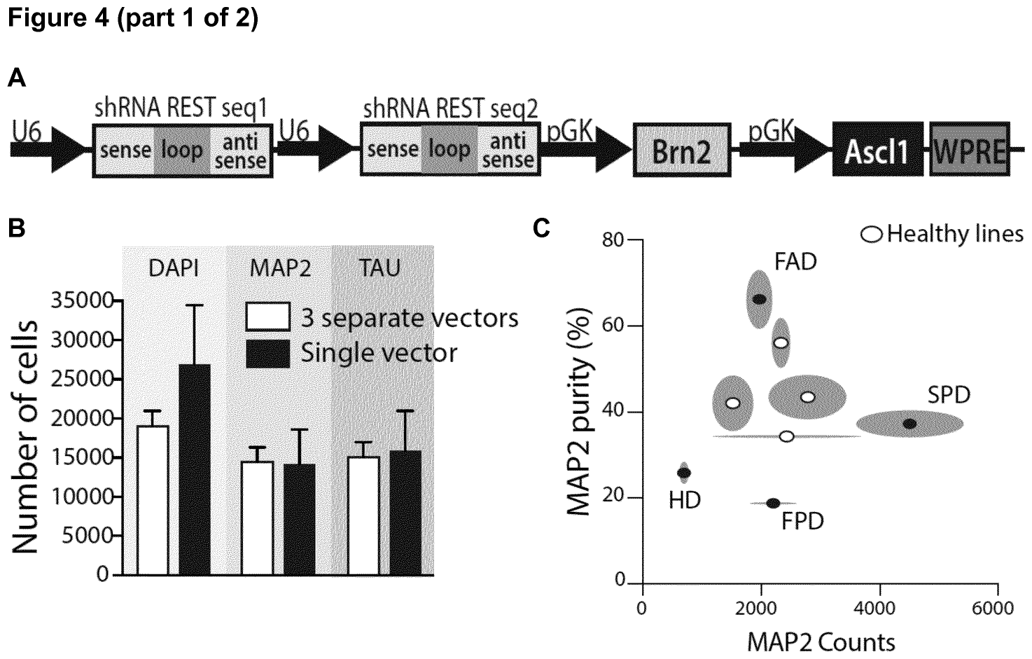

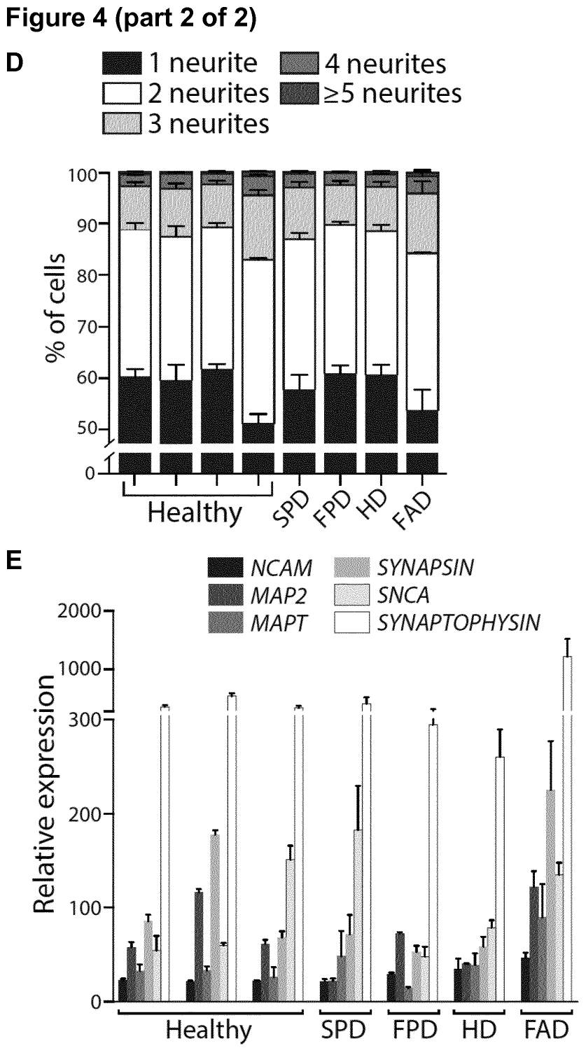

FIG. 4. All-in-one vector to reprogram skin fibroblasts from patients with a range of different neurodegenerative disorders.

(A) Map of the single reprogramming vector containing REST shRNA sequences as well as Brn2a and Ascl1. (B) Quantitative comparison of the total number of cells, as well as the number of MAP2⁺ and TAU⁺ cells per well using separate or one single vectors for pB.pA+RESTi reprogramming in four different adult dermal fibroblast lines. (C) Quantification of the neuronal counts and purity. (D) percentage of cells displaying various number of neurites for each line (n=3 replicates per line). (E) qPCR analysis of 6 neuronal genes in healthy individuals as well as from patients with various neurodegenerative disorders. Data information: Abbreviations: FAD: Familial Alzheimer's disease; FPD: Familial Parkinson's disease; HD: Huntington's disease; SPD: sporadic Parkinson's disease. Data are expressed as mean±SEM and are from biological replicates (n=4).

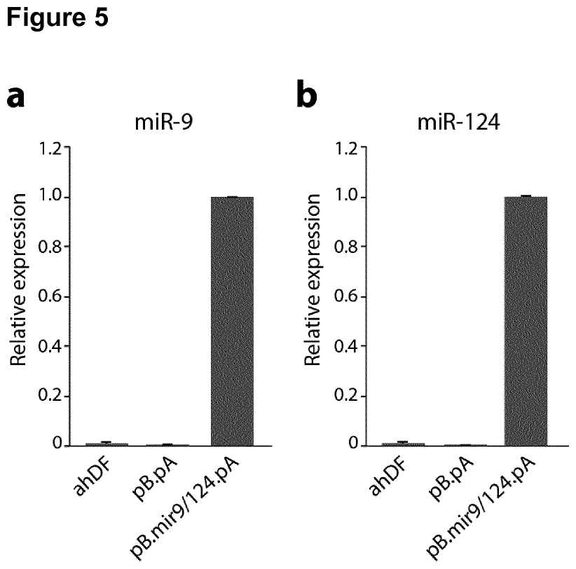

FIG. 5. High miRNA-9 and miRNA-124 expression following transduction with pB.mir9/124.pA. (a, b) Quantitative PCR analysis of miR-9 (a) and miR-124 (b) three days following the transduction with either pB.pA or pB.mir9/124.pA as compared to fibroblast levels. Abbreviations: ahDF: adult human dermal fibroblasts.

### EXAMPLE 1 REST SUPPRESSION MEDIATES NEURAL CONVERSION OF ADULT HUMAN FIBROBLASTS VIA MICRORNA-DEPENDENT AND -INDEPENDENT PATHWAYS

### Introduction

Direct conversion of human fibroblasts into mature and functional neurons, termed induced neurons (iNs), was achieved for the first time 6 years ago. This technology offers a promising shortcut for obtaining patient- and disease-specific neurons for disease modeling, drug screening, and other biomedical applications. However, fibroblasts from adult donors do not reprogram as easily as fetal donors, and no current reprogramming approach is sufficiently efficient to allow the use of this technology using patient-derived material for large-scale applications. Here, we investigate the difference in reprogramming requirements between fetal and adult human fibroblasts and identify REST as a major reprogramming barrier in adult fibroblasts. Via functional experiments where we overexpress and knockdown the REST-controlled neuron-specific microRNAs miR-9 and miR-124, we show that the effect of REST inhibition is only partially mediated via microRNA up-regulation. Transcriptional analysis confirmed that REST knockdown activates an overlapping subset of neuronal genes as microRNA overexpression and also a distinct set of neuronal genes that are not activated via microRNA overexpression. Based on this, we developed an optimized one-step method to efficiently reprogram dermal fibroblasts from elderly individuals using a single-vector system and demonstrate that it is possible to obtain iNs of high yield and purity from aged individuals with a range of familial and sporadic neurodegenerative disorders including Parkinson's, Huntington's, as well as Alzheimer's disease.

### Results

### Development of a Bicistronic Vector for Co-Delivery of Neural Conversion Genes

In order to achieve a highly effective and reproducible conversion system with less variability in transcription factor expression in each cell, we generated and tested three different dual promoter vectors. Although the level of expression of each transgene may vary between each cell, this dual vector approach insures a delivery of the coding sequence of the two neural conversion genes Ascl1 (NM_008553.4) and Brn2 (NM_008899.2) in all cells. The vectors are based on the human PGK promoter and the conversion genes were placed in a different order and distance from the Woodchuck Hepatitis Virus Posttranscriptional Regulatory Elements (WPRE) (FIG. 1a). For a regulatable system the PGK promoter can be replaced by a doxycycline regulatable promoter. When expressed in human fetal fibroblasts, the three constructs resulted in different levels of expression of the conversion genes (FIG. 1b), and we found that the pB.pA construct, yielding the highest ASCL1 to BRN2 protein expression ratio, resulted in the highest level of neural conversion. When co-delivering the two conversion factors using the pB.pA dual promoter vector we found that we increased the yield of iNs by more than 30 fold compared to when the neural conversion factors were delivered using separate vectors (FIG. 1c).

### Difference in Conversion Mechanism/Requirement Between Fetal and Adult Fibroblasts

Global gene expression analysis confirmed that the pB.pA dual promoter construct induced a major change in gene expression in the fetal fibroblasts. We found 561 significantly (Benjamini-Hochberg (BH) corrected p-value <0.001) up-regulated and 328 significantly down-regulated genes 5 days after delivering the conversion vector. Gene ontology analysis showed that many of the up-regulated genes were associated with a neuronal identity, in line with the high conversion yield observed using this reprogramming vector. We next used the same system to convert adult human dermal fibroblasts from a healthy 67-year-old individual. However, we detected only very few, if any, iNs after 30 days. To rule out the possibility that this failure to reprogram was in fact related to adult vs. fetal fibroblasts and not due to difference in the origin of the fibroblast, we confirmed the failure to reprogram using adult lung fibroblasts from a 45-65 individual.

To better understand the difference in reprogramming requirements between fetal and adult fibroblasts, we assessed the transcriptional response in the cells after delivery of the dual-conversion vector using RNA-seq. We found that while 204 genes were up-regulated (p<0.001) in both adult and fetal fibroblasts after transduction with pB.pA dual promoter vector, another 357 and 421 genes were uniquely up-regulated in the transduced fetal or adult fibroblasts respectively (Pearson correlation: 0.307, FIG. 1d). GO analysis of the genes up-regulated in the fetal, but not adult fibroblasts resulted in gene categories associated with neuronal functions (FIG. 1e). This demonstrates that the neural conversion factors activates a largely different set of genes with limited overlap in the two starting populations, and suggests that there are specific barriers to reprogramming present in adult but not fetal fibroblasts. When looking at the top 10 genes related to neuronal differentiation and development uniquely up-regulated in the fetal fibroblasts, 4 were identified as REST targets: JAG2, L1CAM, DYNLL2, DCLK1, suggesting that REST blocks the activation of neuronal genes and subsequent neuronal conversion in the adult fibroblasts.

### REST Inhibition Removes Neural Reprogramming Block in Human Adult Lung and Dermal Fibroblasts

To test the hypothesis that REST prevents neural conversion of adult fibroblasts transduced with ASCL1 and BRN2, we performed qRT-PCR analysis in fetal and adult fibroblasts which revealed slightly increased levels of REST transcripts in adult cells (FIG. 2a, p<0.05). We next used RNAi to knockdown REST, which reduced REST transcript levels in adult fibroblasts down to that observed in fetal human fibroblasts (FIG. 2a). When we expressed the dual promoter conversion vector together with the shRNAs against REST in adult dermal fibroblasts from two different donors (age 61 and 67) we consistently observed exceptionally high neural conversion levels. We also confirmed that RESTi removes the reprogramming barrier also of adult lung fibroblasts. The high conversion efficiency was confirmed using five primary lines from dermal biopsies of individuals aged from 61 to 71 years old and sourced from three different clinical sites (FIG. 2b). We also observed that in contrast to previous reports demonstrating that the reprogramming efficiency decreases at higher passages, there was no decrease in the conversion efficiency or neuronal purity when the fibroblasts from a 67-year-old donor were reprogrammed with the dual promoter construct and RESTi at passages ranging from 3 to 10 (FIG. 2c) implying that RESTi also removes the barriers to reprogramming associated with extensive passaging of the fibroblasts previously observed.

We next analyzed the mature neuronal properties of the resulting iNs. We found that they did indeed express mature neuronal markers such as MAP2, NEUN, SYNAPSIN and TAU. Patch clamp electrophysiological recordings of the iNs after terminal differentiation and maturation in culture showed that they had acquired the functional properties of neurons (FIG. 2d and Table S2). This was also the case when cells pre-labeled with a vector containing GFP expressed under the control of the human synapsin promoter were transplanted to the neonatal brain and analyzed after 7-9 weeks of maturation in vivo. When analyzing the transplanted iNs detected based on GFP expression, we again found current evoked multiple action potentials in the iNs (n=8 from 4 different mice) (FIG. 2e), and the cells displayed postsynaptic currents that could be blocked with the glutamate antagonist CNQX (FIG. 2e), demonstrating that these adult iN cells converted in the presence if RESTi functionally mature, integrate and receive glutamatergic synaptic inputs from the host brain.

RESTi Results in Up-Regulation of Neural Specific miRNAs

MiRNAs have been implicated as important mediators of cell reprogramming, including in neural conversion. Inhibition of REST is known to increase expression of neuron specific miRNAs, and we speculated that the potential up-regulation of miRNAs could be what mediated the effect of RESTi during neural conversion of adult human fibroblast. We therefore assessed the neuron specific miRNA expression levels in the absence and presence of REST inhibition, and found that miR-9 was up-regulated when adult fibroblasts are converted in the presence of RESTi (FIG. 3a). We also checked the expression of a number of region-specific miRNAs but found no clear differences, indicating that RESTi affects pan-neuronal expression without affecting subtype identity (FIG. 3b). To further investigate this, we tested if expression of neuron specific miRNAs could mimic the effect of RESTi. We therefore expressed miR-9/9* and miR-124 together with the conversion factors (FIG. 3c) but without RESTi. We found that adult fibroblasts transduced with this construct expressed high levels of miR-9 and miR-124 (FIG. S1a, b) and converted adult fibroblasts into neurons with similar efficiency to the cells treated with RESTi (FIG. 3d), supporting the hypothesis that RESTi effect could be mediated via up-regulation of miR-9/9* and miR-124, and that miRNAs, like RESTi removes the reprogramming barrier in adult fibroblasts allowing also fibroblasts from aged donors to efficiently and reproducibly be converted into neurons.

To experimentally address whether the RESTi effect is mediated via miRNA up-regulation, we next performed conversions using pB.pA+RESTi while simultaneously knocking down miR-124 or miR-9 in the cells and checked for effects on neural conversion (FIG. 3e-f). We found that while inhibition of miR-124 during the conversion did not significantly affect the iN formation (FIG. 3e), the inhibition of miR-9 during the conversion resulted in a decrease in the number of iNs generated compared to control (FIG. 3f).

Taken together, our data show that the effect of RESTi can be mimicked via miRNA overexpression but that blocking miRNA inhibition during the conversion process only partially affects the neural conversion. This supports that the RESTi acts via miRNA activation and the previously suggested interplay between RESTi and miRNAs.

### MicroRNA Independent Effects of REST Inhibition

In order to better understand the mechanisms that mediate the conversion of adult fibroblasts driven by RESTi or miR-9/miR-124, we performed a comparative global gene expression analysis using RNA sequencing 5 days following the initiation of conversion. In this analysis, we included unconverted adult human fibroblasts and adult fibroblasts in which REST is inhibited as controls. The conversion groups included were: pB.pA (that gives rise to only very low level iN conversion if any); pB.pA+RESTi; pB.miR9/124.pA and pB.miR9/124.pA+RESTi. We compared the genes up-regulated (BH-corrected p-value <0.001) in the pB.pA+RESTi group and the pB.miR9/124.pA groups. This analysis showed that both RESTi and miR-9/miR-124 delivery caused a major transcriptomic change in the cells, and that the effect was not cumulative. Further analysis showed that most of the genes with the largest FC are significant in both the miR-9/miR-124 and RESTi transduced cells (Pearson correlation=0.81). Most genes (more than 1700) were up-regulated in both groups suggesting that these factors largely work on the same neurogenic pathway(s) and activate similar gene cascades.

We next investigated in more detail the differences in gene expression profiles between the RESTi- and miRNA-converted cells. Unsupervised clustering based on euclidean sample distances revealed that the two controls (fibroblasts and fibroblasts+RESTi) as well as the pB.pA (very low conversion group) clustered together while all three groups with successful neural conversion clustered together. Principal component analysis revealed that the three conversion groups were very similar on the PC1 axis, and distinctly different from the control groups. Furthermore, the PC2 axis showed a separation of the groups with RESTi, from those without. The GO term and Kyoto Encyclopedia of Genes and Genomes (KEGG) pathway analyses of the differentially expressed genes revealed that those differentially expressed in the RESTi conversion group were enriched for the regulation of synaptic transmission, synaptic plasticity as well as cell morphogenesis and the differentiation and regulation of neurogenesis and synapse formation. In contrast, the genes uniquely up-regulated in the pB.miR9/124.pA were not associated with neuronal properties

Taken together, our results show that the RESTi, when combined with the neural conversion genes Ascl1 and Brn2a, overcomes human specific barriers of both reprogramming and neuronal maturation. The miRNA knockdown experiments, as well as the global transcriptome analysis, suggest that this effect is only partially mediated via miR-9/miR-124 expression.

Based on this, we designed and cloned a single “all-in-one” construct that expressed both RESTi hairpins and conversion genes on the same construct (FIG. 4a). This vector resulted in similar conversion efficiencies compared to the vector system in which the conversion genes are delivered using the dual promoter vector pB.pA and the two REST shRNAs on three separate vectors while less virus is needed (FIG. 4b). Modeling neurodegenerative disorders would greatly benefit from this technology, as iNs from elderly donors have been shown to maintain their aging signature, which is critical given that age is the biggest risk factor for developing these disorders. To establish its utility for generating cells for disease modeling, we used the new single vector system to convert dermal fibroblasts from healthy adults as well as individuals with sporadic PD, familial PD (LRRK2 c.6055G>A mutation), HD (41 CAG repeats) and familial AD (APP KM670/671NL mutation) (Table S1). All lines were successfully converted to iNs expressing MAP2, albeit with some variation between the lines in terms of yield and purity (FIG. 4c). We also used TAU as a neuronal marker in addition to MAP2 in order to assess the conversion into more mature neurons. Conversion of all lines resulted in neurons with a similar morphological complexity as assessed by the proportion of cells developing variable numbers of neurites for each line (FIG. 4d). Additionally, qPCR analysis revealed a major increase in all the neuronal genes that we assessed (NCAM, MAP2, MAPT, SYNAPSIN, SNCA and SYNAPTOPHYSIN) in every line converted, independently of the disease status of the donor (FIG. 4e).

TABLE 1
Demographic information on the biopsy donors

| | | | | | Disease | |
|---|---|---|---|---|---|---|
| | | | | | Duration | |
| Cells source | Source | Disease | Sex | Age | (years) | Mutation |
| Dermal biopsy | John van Geest | None | F | 52 | | |
| | Centre for | | | | | |
| | Brain Repair | | | | | |
| Dermal biopsy | John van Geest | None | F | 61 | | |
| | Centre for | | | | | |
| | Brain Repair | | | | | |
| Dermal biopsy | John van Geest | None | F | 67 | | |
| | Centre for | | | | | |
| | Brain Repair | | | | | |
| Dermal biopsy | John van Geest | None | M | 69 | | |
| | Centre for | | | | | |
| | Brain Repair | | | | | |
| Dermal biopsy | John van Geest | None | F | 70 | | |
| | Centre for | | | | | |
| | Brain Repair | | | | | |
| Dermal biopsy | John van Geest | None | M | 71 | | |
| | Centre for | | | | | |
| | Brain Repair | | | | | |
| Lung biopsy | Lund | None | F | 45-65 | | |
| | University, | | | | | |
| | Lund | | | | | |
| Dermal biopsy | Lund | None | F | 74 | | |
| | University, | | | | | |
| | Lund | | | | | |
| Dermal biopsy | Karolinska | Genetic | F | 58 | 4 | APP |
| | Institute, | Alzheimer's | | | | KM670/671NL |
| | Stockholm | disease | | | | |
| Dermal biopsy | John van Geest | Huntington's | M | 61 | | HTT - 41 CAG |
| | Centre for | disease (41 | | | | repeats |
| | Brain Repair | CAG repeats) | | | | |
| Dermal biopsy | John van Geest | Sporadic | M | 77 | 4 | |
| | Centre for | Parkinson's | | | | |
| | Brain Repair | disease | | | | |
| Dermal biopsy | John van Geest | Genetic | F | 55 | 8 | LRRK2 |
| | Centre for | Parkinson's | | | | c.6055G > A |
| | Brain Repair | disease | | | | mutation |

TABLE 2
Summary of the electrophysiological properties

| Intrinsic properties | Adult iN in vitro | | Adult iN in vivo | |
|---|---|---|---|---|
| Resting membrane potential | −47.28 ± | 4.05 | −71.14 ± | 3.93 |
| (mV) | (n = 18) | | (n = 7) | |
| Cell capacitance (pF) | 21.78 ± | 7.55 | 60.88 ± | 11.99 |
| | (n = 22) | | (n = 8) | |
| Membrane resistance (MΩ) | 1808 ± | 308.3 | 76.63 ± | 33.89 |
| | (n = 20) | | (n = 8) | |
| Number of AP able to evoke | 1.35 ± | 0.65 | 3.25 ± | 1.35 |

| | | |
|---|---|---|
| | (n = 20) | (n = 8) |
| N of cells with post synaptic | 1/20 | 6/8 |
| activity | | |

### Discussion

The direct conversion of one cell type to another, without going through a stem cell intermediate, has been successfully achieved for a number of cell types including the generation of neurons. This type of conversion makes it possible to study otherwise hard to access patient and disease specific neurons, and holds great promise for creating age relevant models of neurological disorders. iNs, that are obtained via direct conversion, present a faster route by which to generate neurons compared to conventional reprogramming approaches using induced pluripotent stem cells (iPSCs) followed by directed differentiation. However, as iN technology converts one mature cell type directly into a post-mitotic neuron, the requirement for high yield conversion is absolutely essential in order to obtain a sufficient number of neurons for downstream applications.

To date, over a dozen studies have reported successful neural reprogramming of adult primary dermal fibroblasts using a wide array of conversion genes, chemical cocktails and miRNAs, but all have resulted in relatively low numbers of induced neurons. While purification steps or antibiotic selection can increase the purity of the iNs, this is associated with large cell loss making the total yield low which in turn requires a high number of input cells which in this case is limited since adult dermal fibroblasts do not expand indefinitely. In this study, we set out to gain a better mechanistic understanding of the road blocks to reprogramming present specifically in adult human fibroblast, by studying the early transcriptional response in fetal vs. adult fibroblasts. We found that the most commonly used neural conversion genes (ASCL1 and BRN2) elicit largely distinct transcriptional response in these two populations. Bioinformatic data from our experiments showed that many of the genes that were up-regulated only in the fetal fibroblasts were REST targets and thus suggested REST as a potential adult specific reprogramming barrier.

We thus focused our subsequent studies on the knockdown of REST. RESTi has also been shown to induces the expression of miR-124 as well as miR-9 in a number of cell types which is interesting given that these miRNAs can mediate neural conversion alone or when expressed together with neuronal transcription factors. We also show that while the effect of RESTi can be partially mimicked via overexpression of neuron specific miRNAs, inhibiting activation of miRNA during the neural conversion process only partially inhibits the formation of iNs. This suggests that RESTi mediates its effect on neural conversion both via up-regulation of neuronal miRNAs but also via a miRNA independent mechanism. This hypothesis was supported by our comparative RNA-seq analysis that revealed that while many of the same neuronal genes are up-regulated in fibroblasts converted with RESTi, miRNA overexpression or both RESTi and miRNA expression combined, additional gene transcription changes that are associated with a neuronal identity are uniquely up-regulated when fibroblasts are reprogrammed in the presence of RESTi.

Combined, our results show that a conversion strategy based on co-delivery of the conversion factors Ascl1 and Brn2 in combination with RESTi is sufficient to overcome the reprogramming barriers previously associated with adult donors, in the absence of additional miRNA expression. It results in high efficiency and high purity conversion of aged dermal fibroblasts without the need for a purification step. In addition, we also show that the passage number of the starting fibroblast culture does not impact on the reprogramming efficiency, at least up until 10 passages, ensuring that one skin biopsy will provide enough iN material to complete large scale disease modeling, drug screening and transplantation studies. For example, with the efficiency of our system it would be possible to obtain approximately 10 billion neurons from one skin biopsy, which by far makes our method the most efficient approach reported to date using skin biopsies from elderly donors. This makes our approach suitable to explore any potential disease-associated phenotypes in these cells, as well as offering a readily available source of relevant cells for drug screenings and diagnostics.

### Material and Methods

### Biopsy Sampling

Adult dermal fibroblasts were obtained from the Parkinson's Disease Research and Huntington's disease clinics at the John van Geest Centre for Brain Repair (Cambridge, UK) and used under local ethical approval (REC 09/H0311/88); from the Clinical Memory Research Unit (Malmö, Sweden) and used under the Regional Ethical Review Board in Lund, Sweden (Dnr 2013-402); from the Karolinska Institutet (Stockholm, Sweden) (Dnr 2005/498-31/3, 485/02; 2010/1644-32); and lung fibroblasts from a healthy individual with no clinical history of lung disease from Lunds Universitet under approval of the local Ethics committee (Dnr 413/2008 and 412/03) (See Table S1). Written informed consent was taken from each participant and the skin biopsies were taken with a 4 mm punch biopsy from the upper or lower arm under local anesthetic (1% lidocaine), and the site was then closed with steri-strips or a stitch. Primary fibroblast cultures from biopsies were cultured according to the two following methods: 1) fibroblasts were isolated using standard fibroblast medium (Dulbecco's Modified Eagle Medium (DMEM)+Glutamax (Gibco) with 100 mg/mL penicillin/streptomycin (Sigma), and 10% FBS (Biosera)). The skin biopsy was sectioned into 4-6 pieces and placed in a 6 cm dish coated with 0.1% gelatin containing 1.5 ml of medium, which was topped up with 0.5 ml every 2-3 days for a week. One week after the initial plating down of the cells, all of the medium was removed and 2 ml of fresh medium was added. Medium was changed every 3-4 days until full confluency of the fibroblasts was observed. The skin biopsy specimen was then transferred into a new dish and the process was repeated until no more cells grew out of the biopsy. 2) Subjects from the Swedish Biofinder Study had a 3 mm skin punch biopsy taken through the whole dermis to the subcutaneous fat layer using standard clinical procedures. The biopsies were immediately placed on ice in phosphate buffered saline containing calcium and magnesium with glucose (1.8 g/l) and antibiotic-antimycotic (Gibco). Within 1.5-4 hours the biopsies were cut into 10-15 pieces avoiding the subcutaneous fat and the epidermis. The dermal pieces were placed in one well of a 6-well culture plate (Nunclon) and left inside a laminar flow cabinet until dry, usually for less than 15 min. 2 ml fibroblast culture medium (DMEM, 20% FBS, penicillin-streptomycin, sodium pyruvate and antibiotic-antimycotic, all from Gibco) was then added. Incubation was in a standard cell culture incubator in 5% CO₂ and humidified air at 37° C. Half the medium was changed twice weekly. When approximately 30% of the culture well surface was covered by fibroblasts cells were harvested by trypsinisation for approximately 5 min at 37° C. (0.05% trypsin/EDTA, Sciencell). Cells were washed, centrifuged for 3 min at 100×g at room temperature, transferred to a T25 culture flask (Nunc) and cultured in either DMEM (as above but with 10% FBS) or in a defined serum free medium (Fibrolife, Lifeline Celltech). The explants were fed with new DMEM with 20% FBS and placed back in the incubator to allow more fibroblasts to migrate out. Fibroblasts expanded in T25 flasks were either transferred to one T75 flask (Nunc) or frozen for long-term storage. For the lung biopsy, alveolar parenchymal specimens were collected 2-3 cm from the pleura in the lower lobes. Vessels and small airways were removed from the peripheral lung tissues and the remaining tissues were chopped into small pieces and allowed to adhere to the plastic of cell culture flasks for 4 h. They were then kept in cell culture medium in 37° C. cell incubators until the outgrowth of fibroblasts was confluent.

### Cell Culture and Cell Lines

HFL1 (ATCC-CCL-153) cells were obtained from the American Type Culture Collection (ATCC), and expanded in standard fibroblast medium. All the fibroblasts used in this study were expanded at 37° C. in 5% CO₂ in fibroblast medium. The cells were then dissociated with 0.05% trypsin, spun, and frozen in either 50/50 DMEM/FBS with 10% DMSO (Sigma) or DMEM+10% FBS with 10% DMSO.

### Viral Vectors and Virus Transduction

DNA plasmids expressing mouse open reading frames (ORFs) for Ascl1 or Brn2 or a combination of Ascl1 and Brn2 with or without short hairpin RNA (shRNA) targeting REST or miRNA loops for miR-9/9* and miR-124 in a third-generation lentiviral vector containing a non-regulated ubiquitous phosphoglycerate kinase (PGK) promoter were generated. For electrophysiological recordings in vivo, a vector expressing GFP under the control of the neuron specific Synapsin promoter was generated and cells were transduced at a multiplicity of infection (MOI) of 5 on day 0. All the constructs have been verified by sequencing. Lentiviral vectors were produced using standard techniques and titrated by quantitative PCR (qPCR) analysis. Unless otherwise stated, transduction was performed at a MOI of 10 for separate vectors and MOI 20 for the single vector (all viruses used in this study tittered between 3×10⁸ and 6×10⁹).

### Neural Reprogramming

For direct neural reprogramming, fibroblasts were plated at a density of 27 800 cells per cm² in 24-well plates (Nunc) coated with 0.1% gelatin (Sigma). Three days after viral transduction, fibroblast medium was replaced by neural differentiation medium (NDiff227; Takara-Clontech) supplemented with growth factors at the following concentrations: LM-22A4 (2 μM, R&D Systems), GDNF (2 ng/mL, R&D Systems), NT3 (10 ng/μL, R&D Systems) and db-cAMP (0.5 mM, Sigma) and the small molecules CHIR99021 (2 μM, Axon), SB-431542 (10 μM, Axon), noggin (0.5 μg/ml, R&D Systems), LDN-193189 (0.5 μM, Axon), as well as valproic acid sodium salt (VPA; 1 mM, Merck Millipore). Half of the neuronal conversion medium was replaced every 2-3 days. Cells were replated onto a combination of polyornithine (15 μg/mL), fibronectin (0.5 ng/μL) and laminin (5 μg/mL) coated 24-well plates at day 12 post-transduction. 18 days post-transduction, the small molecules were stopped and the neuronal medium was supplemented with only the growth factors (LM-22A4, GDNF, NT3 and db-cAMP) until the end of the experiment.

microRNA Knockdown Experiment

Eight tandem repeats of an imperfectly complementary sequence, forming a central bulge when binding to miR-9 and miR-124 (knock down sponge sequence), were synthesized and cloned into a third-generation lentiviral vector under a PGK promoter. The sponge sequences were as follow: miR-9 TATCATACAGCTACGACCAAAGACG (SEQ ID NO: 5) and miR-124 TGGCATTCATACGTGCCTTAA (SEQ ID NO: 6). Adult dermal fibroblasts were transduced with lentiviral vectors containing pgk.Brn2a.pgk.Ascl1 (pB.pA), REST shRNA (all MOI=10) and either mCherry.mir-9.sp and GFP.mir-124.sp or control vectors containing the reporter gene only (mCherry or GFP) (All MOI=5). Cells were transduced again weekly with the mCherry.mir-9.sp, GFP.mir-124.sp, mCherry or GFP and triplicates of each conditions were analyzed at 25 days post-transduction with the reprogramming factors. Average fluorescence intensity analysis was performed on GFP⁺ or mCherry⁺ cells.

### Immunocytochemistry, Imaging and High Content Screening Quantifications

Cells were fixed in 4% paraformaldehyde, permeabilized with 0.1% Triton-X-100 in 0.1 M PBS for 10 min. Thereafter, cells were blocked for 30 min in a solution containing 5% normal serum in 0.1 M PBS. The following primary antibodies were diluted in the blocking solution and applied overnight at 4° C.: mouse anti-ASCL1 (1:100, BD Biosciences), goat anti-BRN2 (1:500, Santa Cruz Biotechnology), rabbit anti-MAP2 (1:500, Millipore), mouse anti-MAP2 (1:500, Sigma), mouse anti-NEUN (1:100, Millipore), rabbit anti-SYNAPSIN I (1:200, Calbiochem), mouse anti-TAU clone HT7 (1:500, Thermo Scientific) and rabbit anti-TUJ1 (1:500, Covance). Fluorophore-conjugated secondary antibodies (Jackson ImmunoResearch Laboratories) were diluted in blocking solution and applied for 2 hrs. Cells were counterstained with DAPI for 15 min followed by three washes in PBS. The total number of DAPI⁺, MAP2⁺ and TAU⁺ cells per well as well as the average fluorescence intensity for ASCL1, BRN2 and TAU were quantified using the Cellomics Array Scan (Array Scan VTI, Thermo Fischer). Applying the program “Target Activation”, 289 fields (10× magnification) were acquired in a spiral fashion starting from the center. The same array was used for the analysis of the number of neurites per TAU⁺ cells using the program “Neuronal Profiling”. Neuronal purity was calculated as the number of MAP2⁺ or TAU⁺ over the total number of cells in the well at the end of the experiment, whereas conversion efficiency was calculated as the number of TAU⁺ over the total number of fibroblasts plated for reprogramming.

### Fluorescence Activated Cell Sorting

For qRT-PCR analysis of neuronal gene expression, reprogrammed cells were detached from cultureware with Accutase (PAA Laboratories), gently triturated and washed with washing buffer containing Hank's balanced salt solution (GIBCO) with 1% bovine serum albumin and DNAse. Fibroblasts were either directly used for sorting according to GFP expression or incubated in washing buffer containing a mouse anti-human NCAM antibody labeled with APC (1:50 for fetal fibroblasts or 1:10 for adult fibroblasts, BD Biosciences) for 15 min at 4° C. The cells were sorted using a FACSAria III cell sorter according to human NCAM (Neural cell adhesion molecule 1) expression gated against unstained converted iNs.

qRT-PCR Analysis for miR-9, miR-124 and RE1-Silencing Transcription Factor

Total RNA, including miRNA, was extracted from human fibroblasts as well as NCAM⁺ sorted converted fibroblasts from the same lines using the micro miRNeasy kit (Qiagen) followed by Universal cDNA synthesis kit (Fermentas, for RNA analysis; Exiqon for miRNA expression). Three reference genes were used for each qPCR analysis (ACTB, GAPDH and HPRT1). LNA-PCR primer sets, specific for hsa-miR-9-5p, hsa-miR-124-3p and hsa-miR-103 (the latter used as normalization miRNA), were purchased from Exiqon and used for the miRNA qPCR analysis. All primers were used together with LightCycler 480 SYBR Green I Master (Roche). Standard procedures of qRT-PCR were used, and data quantified using the ΔΔCt-method. Statistical analyses were performed on triplicates from each groups.

### RNA-Seq Analysis

Fibroblasts were transduced with the different lentiviral vectors (pB.pA or pB.mir9/124.pA+/−RESTi) and both untransduced fibroblasts and fibroblasts transduced only with REST shRNA were used as controls (CTR). Cells were collected 5 days after transduction. RNA was extracted using RNAeasy mini kit (Qiagen) with DNase treatment and sent for RNA-seq to UCLA Clinical Microarray Core. cDNA libraries were prepared using the KAPA Stranded mRNA-Seq Kit from KAPAbiosystems. The 50-bp single-end reads from the Illumina HiSeq 2000 were mapped to the human genome assembly (GRCh38) using STAR (2.4.0j) with default parameters. mRNA expression was quantified using the subread package FeatureCounts quantifying to NCBI annotation (GRCh38). Read counts were normalized to the total number of reads mapping to the genome. Clustering and differential expression analysis was done with DESeq2. Downstream analyses were performed using in-house R and unix scripts. Gene ontology analysis was done with the Functional Annotation Tool of DAVID Bioinformatic Resources 6.7. To get a list of uniquely up-regulated genes in the gene ontology analysis BH-corrected p-values<0.001 were used to get the genes strongly up-regulated in one group (fetal fibroblasts+pB.pA and pB.pA+RESTi), while genes with p-value <0.05 in the other group (adult fibroblasts+pB.pA and pB.mir9/124.pA) were removed from the gene list. This ensured that no genes that showed a strong trend for up-regulation were classified as “not up-regulated”. For the principal component analysis (PCA) one of the pB.pA+RESTi triplicate clustered with the pB.pA group which is most likely due to lack of co-expression of pB.pA and REST shRNA as they are delivered on separate vectors. This group was excluded from further analysis.

### Transplantation

Adult fibroblasts were first transduced with Syn-GFP and then lentiviral vectors containing pB.pA, REST shRNAs. Cells were prepared for transplantation 3 days past initiation of neural conversion and transplanted to the striatum of neonatal rats (p1) under Fentanyl-Dormitor anesthesia using a 5-μL Hamilton syringe fitted with a glass capillary (outer diameter 60-80 μm). The rats received a 1 μL injection of 200 000 cells through one needle penetration. After injection, the syringe was left in place for 2 min before being retracted slowly.

### Electrophysiology

In vitro patch-clamp electrophysiology was performed on iNs reprogrammed from adult dermal fibroblasts on coverslips and co-cultured with glia between day 85 and 100 post-transduction. Cells were recorded in a Krebs solution composed of (in mM): 119 NaCl, 2.5 KCl, 1.3 MgSO₄, 2.5 CaCl₂, 25 Glucose and 26 NaHCO₃. Cells (n=20) with a neuronal morphology as evidenced by them possessing a round cell body, processes and expressing GFP under the control of the synapsin promoter (co-transduced with the reprogramming factors) were patched for whole-cell recordings.

For recordings on slices, coronal brain slices from transplanted rats were prepared at 8 weeks post-conversion. Rats were killed by an overdose of pentobarbital and the brains were rapidly removed and cut coronally on a vibratome at 275 μm. Slices were transferred to a recording chamber and submerged in a continuously flowing Krebs solution gassed with 95% 02 and 5% CO₂ at 28° C. The composition of the Krebs solution for slice recording was (in mM): 126 NaCl, 2.5 KCl, 1.2 NaH₂PO₄—H₂O, 1.3 MgCl₂-6H₂O, and 2.4 CaCl₂.6H₂O. Converted cells were identified by their GFP fluorescence and patched (n=8 in total).

Recordings were made using Multi-clamp 700B (Molecular Devices), and signals were acquired at 10 kHz using pClamp10 software and a data acquisition unit (Digidata 1440A, Molecular Devices). Borosilicate glass pipettes (3-7MΩ) for patching were filled with the following intracellular solution (in mM): 122.5 potassium gluconate, 12.5 KCl, 0.2 EGTA, 10 Hepes, 2 MgATP, 0.3 Na₃GTP and 8 NaCl and adjusted to pH 7.3 with KOH as in (29). Resting membrane potentials were monitored immediately after breaking into the cell, in current-clamp mode. In cultures, cells were kept at a membrane potential of −60 mV to −80 mV, and 500 ms currents were injected from −20 pA to +90 pA using 10 pA increments to induce action potentials. For slices, action potentials were induced with a 500 ms current injected from −100 pA to +400 pA with 50 pA increments. Spontaneous postsynaptic activity was recorded in current-clamp mode at resting membrane potentials using 0.1 kHz Lowpass filter.

### Statistical Analysis

All data are expressed as mean±the standard error of the mean. Statistical analyses were conducted using the GraphPad Prism 7.0. An alpha level of p<0.05 was set for significance. Groups were compared using a one-way ANOVA with a Bonferroni post hoc or Student t test in case of only two groups.

### EMBODIMENTS OF THE INVENTION

1. A gene expression system comprising
a. A first nucleotide sequence encoding a peptide of Ascl1
b. A second nucleotide sequence encoding a peptide of Brn2
c. A third nucleotide sequence of at least one nucleotide sequence encoding a REST-silencing sequence, such as short hairpin REST sequences suppressing REST-expression
2. According to embodiment 1 where the expression system is a lentiviral vector or any suitable vector system
3. According to any of embodiments above where the nucleotide sequences to be expressed is under the control of a constitutive promoter, such as an PGK promoter or a regulatable promoter
4. According to any of embodiments above where nucleotide sequences of Ascl1 and Brn2 are cloned into the same vector
5. According to any of embodiments above where Ascl1 and Brn2 is cloned to be transcribed into a single transcript (e.g. bicistronic)
6. According to any of the embodiments above the conversion genes were placed in a different order and distance from the Woodchuck Hepatitis Virus Posttranscriptional Regulatory Elements (WPRE) (FIG. 1a)
7. According to any of embodiments above where the order of the first and second nucleotide sequence are pgk.Brn2.pgk.Ascl1 (pB.pA)
8. According to any of embodiments above where gene expression system is comprised in a single vector
9. An mammalian cell transformed/transduced/transfected with the gene expression system of embodiments 1 to 8
10. The mammalian cell of embodiment 9 is a human cell
11. The mammalian cell of embodiment 9 or 10 is a mature cell type cultured to primary fibroblasts
12. The cell of embodiment 9 to 11 is cultured until converted into a post-mitotic neuron directly
13. The cell of embodiments 9 to 12 where the cell is derived from a biopsy sample obtained from an individual animal, such as a human
14. The cell of embodiment 13 where the biopsy sample comprises fibroblasts such as a skin punch biopsy or lung biopsy
15. According to embodiments 9 to 14 where the biopsy sample obtained from an individual with various neurodegenerative disorders, in particular individuals with a history of Familial or sporadic Alzheimer's disease; Familial or sporadic Parkinson's disease; Huntington's disease; or from healthy individuals
16. A method of inducing neurons directly from fibroblast cells comprising the step of transducing said fibroblast cell with the gene expression system of embodiments 1 to 8
17. A method of screening for compounds altering disease related biomarkers comprising the steps of culturing cells of either one of embodiments 9 to 15 comprising the steps of
a. Expose said cells, e.g. induced neurons (iNs) to at least one chemical compound to be tested
b. Register measured levels of a selection of at least one disease related biomarker or intracellular marker
c. Compare the registered measured levels in b. with one or more reference levels
d. Select for compounds altering disease related biomarkers or intracellular markers
18. A method for detecting the presence, progression or early stage onset/development of an age related neurological clinical condition in an individual comprising
a. transduce fibroblasts in a biopsy sample obtained from an individual being investigated, with the gene expression system of embodiments 1 to 7
b. Register measured levels of any potential disease-associated phenotypes or biomarkers in these cells at the stage of induced neuron
c. Compare the registered measured levels in b. with one or more reference levels
d. Stratifying samples based on their correlation to the reference levels in c. as indicative of the absence, the presence, progression or early stage onset/development of an age related neurological clinical condition
19. According to embodiment 18 where the age related neurological clinical condition in an individual is selected from the group comprising Familial and sporadic Alzheimer's disease; Familial and sporadic Parkinson's disease; Huntington's disease
20. Kit of parts for inducing neurons in an animal fibroblast cell, such as a human fibroblast cell comprising
a. An expression vector system according to embodiments 1 to 7
21. Use of either of the embodiments above in diagnostics or for the preparation of biological cells, tissue in cell therapy or for preparing cells or tissue for gene therapy

### Paragraphs of the Invention—I

1. A gene expression system comprising
a. A first nucleotide sequence encoding a peptide of Ascl1
b. A second nucleotide sequence encoding a peptide of Brn2
c. A third nucleotide sequence of at least one nucleotide sequence encoding a REST-silencing sequence, such as short hairpin REST sequences suppressing REST-expression
2. According to paragraph 1 where the expression system is a lentiviral vector or any suitable vector system
3. According to any of paragraphs above where the order of the first and second nucleotide sequence are pgk.Brn2.pgk.Ascl1 (pB.pA)
4. According to any of paragraphs above where gene expression system is comprised in a single vector
5. An mammalian cell transduced with the gene expression system of paragraphs 1 to 4
6. The mammalian cell of paragraph 5 is a human cell
7. A method of inducing neurons directly from fibroblast cells comprising the step of transducing said fibroblast cell with the gene expression system of paragraphs 1 to 4
8. A method of screening for compounds altering disease related biomarkers comprising the steps of culturing cells of either one of paragraphs 5 to 7 comprising the steps of
a. Expose said cells, e.g. induced neurons (iNs) to at least one chemical compound to be tested
b. Register measured levels of a selection of at least one disease related biomarker or intracellular marker
c. Compare the registered measured levels in b. with one or more reference levels
d. Select for compounds altering disease related biomarkers or intracellular markers
9. A method for detecting the presence, progression or early stage onset/development of an age related neurological clinical condition in an individual comprising
a. transduce fibroblasts in a biopsy sample obtained from an individual being investigated, with the gene expression system of paragraphs 1 to 4
b. Register measured levels of any potential disease-associated phenotypes or biomarkers in these cells at the stage of induced neuron
c. Compare the registered measured levels in b. with one or more reference levels
d. Stratifying samples based on their correlation to the reference levels in c. as indicative of the absence, the presence, progression or early stage onset/development of an age related neurological clinical condition
10. Use of any of the paragraphs above in diagnostics or for the preparation of biological material, cells or tissue in cell therapy or for preparing cells or tissue for gene therapy

### Paragraphs of the Invention—II

1. A gene expression system comprising
a. at least one nucleotide sequence encoding a neuronal conversion factor; and
b. at least one nucleotide sequence encoding a REST-silencing sequence capable of suppressing REST-expression.
2. A gene expression system according to paragraph 1 comprising
a. (i) a nucleotide sequence encoding Ascl1;
(ii) a nucleotide sequence encoding Brn2; and
   b. at least one nucleotide sequence encoding a REST-silencing sequence capable of suppressing REST-expression.
   3. A gene expression system according to paragraph 2, wherein the nucleotide sequences of (a) (i) and (a) (ii) are comprised in a single vector.
   4. A gene expression system according to any of paragraphs 1-3, wherein the nucleotide sequences of (a) and (b) are comprised in a single vector.
   5. A gene expression system according to any of paragraphs 1-4, wherein the expression system is a lentiviral vector.
   6. A gene expression system according to any of paragraphs 1-5, wherein the nucleotide sequences of (a), e.g. the nucleotide sequences of (a) (i) and (a) (ii), are configured such that they are transcribed into a single transcript (e.g. bicistronic).
   7. A gene expression system according to any of paragraphs 1-6 wherein the nucleotide sequences are under the control of a constitutive promoter such as a PGK promoter or under the control of a regulatable promoter such as a doxycycline regulatable promoter.
   8. A gene expression system according to any one of paragraphs 2-7 wherein the order of the nucleotide sequences of (a) (i) and (a) (ii) is pBrn2.pAscl1, optionally wherein the promoter is PGK and the order is pgk.Brn2.pgk.Ascl1 (pB.pA).
   9. A gene expression system according to any one of paragraphs 1-8, wherein the gene expression system further comprises a transcriptional regulatory element such as a Woodchuck Heptatitis Virus Posttranscriptional Regulatory Element (WPRE).
   10. A gene expression system according to any one of paragraphs 1-9, wherein the REST-silencing sequence is selected from the group consisting of shRNA, siRNA and miRNA.
   11. A cell comprising the gene expression system of paragraphs 1 to 10, optionally wherein the host cell is mammalian.cell.
   12. The cell of paragraph 11 which is a human cell
   13. The cell of paragraph 11 or 12, wherein the cell is a primary fibroblast that has been cultured from a mature cell type.
   14. The cell of any one of paragraphs 11-13, wherein the cell is derived from a biopsy sample obtained from an animal such as a human.
   15. The cell of paragraph 14, wherein the biopsy sample comprises fibroblasts, such as a skin punch biopsy or a lung biopsy.
   16. The cell of paragraph 14 or 15 wherein the biopsy sample is obtained from an individual with a neurodegenerative disorder, optionally wherein the neurodegenerative disorder is familial or sporadic Alzheimer's disease or familial or sporadic Parkinson's disease, or Huntington's disease; or wherein the biopsy sample is obtained from a healthy individual.
   17. The cell of any one of paragraphs 11-16, wherein following introduction of the gene expression system, the cell has been cultured until converted into an induced neuron directly.
   18. The cell according to paragraph 17, wherein the cell was passaged at least 3 times before introduction of the gene expression system.
   19. A method of inducing neurons directly from somatic cells (eg fibroblast cells) comprising the step of introducing the gene expression system of paragraphs 1 to 10 into a somatic cell (eg fibroblast cell).
   20. A method according to paragraph 19, wherein the gene expression system is introduced into the somatic cell (eg fibroblast cell) by transduction.
   21. A method of paragraph 19 or 20, wherein following introduction of the gene expression system into the somatic cell (eg fibroblast cell), the cells are cultured in a neural differentiation medium, such as NDiff227.
   22. A method according to paragraph 21, wherein the neural differentiation medium is supplemented with one or more growth factors, optionally wherein the one or more growth factors are selected from LM-22A4, GDNF, NT3 and db-cAMP.
   23. A method according to paragraph 21 or 22, wherein the neural differentiation medium is supplemented with one or more small molecules, optionally wherein the one or more small molecules are selected from CHIR99021, SB-431542, noggin, LDN-193189 and valproic acid sodium salt.
   24. A method according to any of paragraphs 19-23, wherein the method further comprises assessing the cell for one or more neuronal characteristics, optionally by at least one method selected from immunocytochemistry, fluorescence activated cell sorting, and electrophysiology.
   25. An induced neuron cell obtainable by carrying out the method of any one of paragraphs 19-24.
   26. A neuronal cell according to paragraph 25, wherein the cell was passaged at least
   3 times, or wherein the cell was passaged up to 50 times before introduction of the gene expression system.
   27. Use of a gene expression system according to any one of paragraphs 1-10, or a cell as defined in any one of paragraphs 11-18, 25 and 26 in disease modelling, or in diagnostics or in drug screening.
   28. A gene expression system according to any one of paragraphs 1-10 or a cell as defined in any one of paragraphs 11-18, 25 and 26 for use in medicine.
   29. A gene expression system according to any one of paragraphs 1-10 or a cell as defined in any one of paragraphs 11-18, 25 and 26 for use in diagnostics, or cell therapy or gene therapy.
   30. A pharmaceutical composition comprising a gene expression system according to any one of paragraphs 1-10 or a cell as defined in any one of paragraphs 11-18, 25 and
   26, and a pharmaceutically acceptable carrier.
   31. A method of screening for a compound that alters at least one disease related biomarker, the method comprising
   a. exposing an induced neuron as defined in any one of paragraphs 17, 18, 25 and
   26 to at least one chemical compound to be tested
   b. registering the level of at least one disease related biomarker
   c. comparing the registered level of at least one disease related biomarker in b. with one or more reference levels; and
   d. selecting at least one compound that alters the level of at least one disease related biomarker with the one or more reference levels.
   32. A method according to paragraph 31, wherein the disease related biomarker is a biomarker of a neurological disorder, such as any of Alzheimer's disease, Parkinson's disease or Huntington's disease.
   33. A method for detecting the presence, progression or early stage onset/development of an age related neurological clinical condition in an individual comprising
   a. introducing the gene expression system of any one of paragraphs 1 to 10 into fibroblasts in a biopsy sample obtained from the individual;
   b. registering the level of at least one potential disease-associated phenotype or biomarker in these cells at the stage of induced neuron
   c. comparing the registered level of at least one potential disease-associated phenotype or biomarker in b. with one or more reference levels; and
   d. stratifying the sample based on the correlation to the reference levels in c. as indicative of the absence, the presence, progression or early stage onset/development of an age related neurological clinical condition.
   34. A method according to paragraph 33, wherein the potential disease-associated phenotype or biomarker is a potential neurological disease-associated phenotype or biomarker. such as any of Alzheimer's disease, Parkinson's disease or Huntington's disease.
   35. A method according to paragraph 33 or 34 wherein the age related neurological clinical condition in an individual is selected from the group comprising Familial and sporadic Alzheimer's disease; Familial and sporadic Parkinson's disease; Huntington's disease.
   36. Use of RESTi in directly converting a fibroblast into an induced neuron.
   37. A method of directly converting a fibroblast into an induced neuron comprising contacting the fibroblast with a REST inhibitor.

1. A gene expression system comprising:
(a) a nucleotide sequence encoding Asci1;
(b) a nucleotide sequence encoding Brn2; and
(c) at least one nucleotide sequence encoding a REST-silencing sequence capable of suppressing REST-expression.

2. A gene expression system according to claim 1, wherein the nucleotide sequences of (a) and (b) are comprised in a single vector, optionally, wherein the nucleotide sequences of (a), (b) and (c) are comprised in a single vector.

3. A gene expression system according to claim 1, wherein the expression system is a lentiviral vector and/or wherein the nucleotide sequences of (a) and (b) are configured such that they are transcribed into a single transcript.

4. A gene expression system according to claim 1 wherein the nucleotide sequences are under the control of a constitutive promoter such as a PGK promoter or under the control of a regulatable promoter such as a doxycycline regulatable promoter.

5. A gene expression system according to claim 1 wherein the order of the nucleotide sequences of (a) and (b) is pBrn2.pAscll, optionally wherein the promoter is PGK and the order is pgk.Brn2.pgk.Asc11 (pB.pA).

6. A gene expression system according to claim 1, wherein the gene expression system further comprises a Woodchuck Heptatitis Virus Posttranscriptional Regulatory Element (WPRE), and/or wherein the REST-silencing sequence is shRNA.

7. A mammalian cell transformed, transduced or transfected with the gene expression system of claim 1, optionally wherein the cell is a human cell.

8. The cell of claim 7, wherein the cell is derived from a biopsy sample obtained from an animal such as a human, optionally (i) wherein the biopsy sample comprises fibroblasts, such as a skin punch biopsy or a lung biopsy; and/or (ii) wherein the biopsy sample is obtained from an individual with a neurodegenerative disorder, optionally wherein the neurodegenerative disorder is familial or sporadic Alzheimer's disease or familial or sporadic Parkinson's disease, or Huntington's disease; and/or (iii) wherein the biopsy sample is obtained from a healthy individual; and/or (iv) wherein following introduction of the gene expression system, the cell has been cultured until converted into an induced neuron directly, optionally wherein the cell was passaged at least 3 times, or wherein the cell was passaged up to 10 times before introduction of the gene expression system.

9. A method of inducing neurons directly from fibroblast cells comprising the step of introducing the gene expression system of claim 1 into a fibroblast cell,
wherein the gene expression system is introduced into the fibroblast cell by transduction.

10. A method according to claim 9, wherein following introduction of the gene expression system into fibroblast cell, the cells are cultured in a neural differentiation medium, such as NDiff227, optionally:
(a) wherein the neural differentiation medium is supplemented with one or more growth factors, optionally wherein the one or more growth factors are selected from LM-22A4, GDNF, NT3 and db-cAMP; or
(b) wherein the neural differentiation medium is supplemented with one or more small molecules, optionally wherein the one or more small molecules are selected from CHIR99021, SB-431542, noggin, LDN-193189 and valproic acid sodium salt; or
(c) wherein the method further comprises assessing the cell for one or more neuronal characteristics, optionally by at least one method selected from immunocytochemistry, fluorescence activated cell sorting, and electrophysiology.

11. An induced neuron cell obtainable by carrying out the method of claim 9, optionally wherein the cell passaged at least 3 times, or wherein the cell was passaged up to 50 times before introduction of the gene expression system.

12. Use of a gene expression system according to claim 1 in disease modelling, or in diagnostics or in drug screening.

13. A gene expression system according to claim 1 for use in diagnostics, or in cell therapy or in gene therapy.

14. A method of screening for a compound that alters at least one disease related biomarker, the method comprising:
(a) exposing an induced neuron as defined in claim 7 to at least one chemical compound to be tested;
(b) registering the level of at least one disease related biomarker;
(c) comparing the registered level of at least one disease related biomarker in b. with one or more reference levels; and
(d) selecting at least one compound that alters the level of at least one disease related biomarker with the one or more reference levels, optionally wherein the disease related biomarker is a biomarker of a neurological disorder, such as any of Alzheimer's disease, Parkinson's disease or Huntington's disease.

15. A method for detecting the presence, progression or early stage onset/development of an age related neurological clinical condition in an individual comprising:
(a) introducing the gene expression system of claim 1 into fibroblasts in a biopsy sample obtained from the individual;
(b) registering the level of at least one potential disease-associated phenotype or biomarker in these cells at the stage of induced neuron;
(c) comparing the registered level of at least one potential disease-associated phenotype or biomarker in (b) with one or more reference levels; and
(d) stratifying the sample based on the correlation to the reference levels in (c) as indicative of the absence, the presence, progression or early stage onset/development of an age related neurological clinical condition,
optionally (i) wherein the potential disease-associated phenotype or biomarker is a potential neurological disease-associated phenotype or biomarker, such as any of Alzheimer's disease, Parkinson's disease or Huntington's disease; and/or (ii) wherein the age related neurological clinical condition in an individual is selected from the group comprising Familial and sporadic Alzheimer's disease; Familial and sporadic Parkinson's disease; Huntington's disease.


SEQUENCE LISTING (6 sequences)

SEQ ID NO: 1 - protein, 236 aa, Homo sapiens
  ASCL1
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 2 - protein, 443 aa, Homo sapiens
  BRN2
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 3 - protein, 1097 aa, Homo sapiens
  REST
MATQVMGQSSGGGGLFTSSGNIGMALPNDMYDLHDLSKAELAAPQLIMLANVALTGEVNGSCCDYLVGEERQMAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSNSEKFSKTKKSKRKLEVDSHSLHGPVNDEESSTKKKKKVESKSKNNSQEVPKGDSKVEENKKQNTCMKKSTKKKTLKNKSSKKSSKPPQKEPVEKGSAQMDPPQMGPAPTEAVQKGPVQVEPPPPMEHAQMEGAQIRPAPDEPVQMEVVQEGPAQKELLPPVEPAQMVGAQIVLAHMELPPPMETAQTEVAQMGPAPMEPAQMEVAQVESAPMQVVQKEPVQMELSPPMEVVQKEPVQIELSPPMEVVQKEPVKIELSPPIEVVQKEPVQMELSPPMGVVQKEPAQREPPPPREPPLHMEPISKKPPLRKDKKEKSNMQSERARKEQVLIEVGLVPVKDSWLLKESVSTEDLSPPSPPLPKENLREEASGDQKLLNTGEGNKEAPLQKVGAEEADESLPGLAANINESTHISSSGQNLNTPEGETLNGKHQTDSIVCEMKMDTDQNTRENLTGINSTVEEPVSPMLPPSAVEEREAVSKTALASPPATMAANESQEIDEDEGIHSHEGSDLSDNMSEGSDDSGLHGARPVPQESSRKNAKEALAVKAAKGDFVCIFCDRSFRKGKDYSKHLNRHLVNVYYLEEAAQGQE

SEQ ID NO: 4 - DNA, 682 bp, Artificial Sequence
  REST-silencing shRNA
gggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtacttcaataaggacttgctccacacgtgaggagcaagtccttattgaagttttttgaattctcgaccggatcccggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtagtacacattaaccaaatggcacacgtgagccatttggttaatgtgtacttttttgaattctcgacctcgagaagcttgatatcgaattc

SEQ ID NO: 5 - DNA, 25 bp, Artificial Sequence
  miR-9 sponge
tatcatacagctacgaccaaagacg

SEQ ID NO: 6 - DNA, 21 bp, Artificial Sequence
  miR-124 sponge
tggcattcatacgtgccttaa